(19) United States
(12) Patent Application Publication
(10) Pub. No.: US 2024/0362382 A1
(43) Pub. Date: Oct. 31, 2024

(54) SELECTIVE BREEDING ASSISTANCE APPARATUS, SELECTIVE BREEDING ASSISTANCE METHOD, AND STORAGE MEDIUM

(71) Applicant: NEC Corporation, Minato-ku, Tokyo (JP)

(72) Inventors: Yoji MORI, Tokyo (JP); Ayako HOSHINO, Tokyo (JP); Yuya ENDO, Tokyo (JP); Yuuki WATANABE, Tokyo (JP); Naruto YAJIMA, Tokyo (JP)

(73) Assignee: NEC Corporation, Minato-ku, Tokyo (JP)

(21) Appl. No.: 18/681,565

(22) PCT Filed: Sep. 15, 2021
(86) PCT No.: PCT/JP2021/033831

§ 371 Date: Feb. 6, 2024

(51) Int. Cl. G06F 30/27

(52) U.S. Cl. CPC G06F 30/27

(57) ABSTRACT

In order to suitably assist breeding, a selective breeding assistance apparatus (1) includes: a reception section (11) for receiving a request pertaining to breeding; a generation section (12) for generating response information based on the request using a learned model which has learned a relation between a property of an existing variety and a breeding process of the existing variety, the response information including information pertaining to an existing variety that serves as a crossbreeding candidate for developing a new variety; and an output section (13) for outputting the response information.

### TECHNICAL FIELD

The present invention relates to a selective breeding assistance apparatus and the like for generating information pertaining to breeding.

### BACKGROUND ART

Breeding for imparting a favorable character to animals and plants has long been carried out mainly in the fields of agriculture, forestry and animal husbandry. In breeding, the following process is carried out which include: selecting varieties to be crossbred; crossbreeding the selected varieties; and checking whether a new variety thus produced by crossbreeding has an intended character (e.g., see Patent Literature 1 below).

### CITATION LIST

### Patent Literature

### [Patent Literature 1]

Japanese Patent Application Publication Tokukai No. 2013-55963

### SUMMARY OF INVENTION

### Technical Problem

Breeding needs to repeat the above process until a new variety having an intended character is found. Therefore, there is a problem that it takes a lot of labor and a lot of time, and a technique for assisting breeding is demanded.

An example aspect of the present invention is accomplished in view of the above problem, and an example object thereof is to provide a technique for suitably assisting breeding.

### Solution to Problem

A selective breeding assistance apparatus in accordance with an example aspect of the present invention includes: a reception means for receiving a request pertaining to breeding; a generation means for generating response information based on the request using a learned model which has learned a relation between a property of an existing variety and a breeding process of the existing variety, the response information including information pertaining to an existing variety that serves as a crossbreeding candidate for developing a new variety; and an output means for outputting the response information.

A selective breeding assistance method in accordance with an example aspect of the present invention includes: receiving, by a computer, a request pertaining to breeding; generating, by the computer, response information based on the request using a learned model which has learned a relation between a property of an existing variety and a breeding process of the existing variety, the response information including information pertaining to an existing variety that serves crossbreeding candidate for developing a new variety; and outputting, by the computer, the response information.

A selective breeding assistance program in accordance with an example aspect of the present invention causes a computer to carry out: a process of receiving a request pertaining to breeding; a process of generating response information based on the request using a learned model which has learned a relation between a property of an existing variety and a breeding process of the existing variety, the response information including information pertaining to an existing variety that serves as a crossbreeding candidate for developing a new variety; and a process of outputting the response information.

### Advantageous Effects of Invention

According to an example aspect of the present invention, it is possible to suitably assist breeding.

### BRIEF DESCRIPTION OF DRAWINGS

FIG. 1 is a block diagram illustrating a configuration of a selective breeding assistance apparatus in accordance with a first example embodiment of the present invention.

FIG. 2 is a flowchart illustrating a flow of a selective breeding assistance method in accordance with the first example embodiment of the present invention.

FIG. 3 is a diagram illustrating learning of feature quantities in graph-based relationship learning.

FIG. 4 is a diagram illustrating an overview of a selective breeding assistance method in accordance with a second example embodiment of the present invention.

FIG. 5 is a block diagram illustrating a configuration of a selective breeding assistance apparatus in accordance with the second example embodiment of the present invention.

FIG. 6 is a flowchart illustrating a flow of a process carried out by the selective breeding assistance apparatus in accordance with the second example embodiment of the present invention.

FIG. 7 is a diagram illustrating an example of response information.

FIG. 8 is a diagram illustrating an overview of a selective breeding assistance method in accordance with a third example embodiment of the present invention.

FIG. 9 is a block diagram illustrating a configuration of a selective breeding assistance apparatus in accordance with the third example embodiment of the present invention.

FIG. 10 is a flowchart illustrating a flow of a process carried out by the selective breeding assistance apparatus in accordance with the third example embodiment of the present invention.

FIG. 11 is a diagram illustrating an overview of a selective breeding assistance method including a process of identifying a similar variety of a base variety.

FIG. 12 is a flowchart illustrating a flow of a process carried out by a selective breeding assistance apparatus when identifying a similar variety of a base variety.

FIG. 13 is a diagram illustrating an overview of a selective breeding assistance method in accordance with a fourth example embodiment of the present invention.

FIG. 14 is a block diagram illustrating a configuration of a selective breeding assistance apparatus in accordance with the fourth example embodiment of the present invention.

FIG. 15 is a flowchart illustrating a flow of a process carried out by the selective breeding assistance apparatus in accordance with the fourth example embodiment of the present invention.

FIG. 16 is a diagram illustrating an overview of a selective breeding assistance method in accordance with a fifth example embodiment of the present invention.

FIG. 17 is a block diagram illustrating a configuration of a selective breeding assistance apparatus in accordance with the fifth example embodiment of the present invention.

FIG. 18 is a flowchart illustrating a flow of a process carried out by the selective breeding assistance apparatus in accordance with the fifth example embodiment of the present invention.

FIG. 19 is a diagram illustrating an example of predicting a character of a new variety based on a feature quantity calculated from a new variety graph and an existing variety graph.

FIG. 20 is a configuration diagram for realizing a selective breeding assistance apparatus by software.

### EXAMPLE EMBODIMENTS

### First Example Embodiment

The following description will discuss a first example embodiment of the present invention in detail, with reference to the drawings. The present example embodiment is a basic form of example embodiments described later.

### (Selective Breeding Assistance Apparatus)

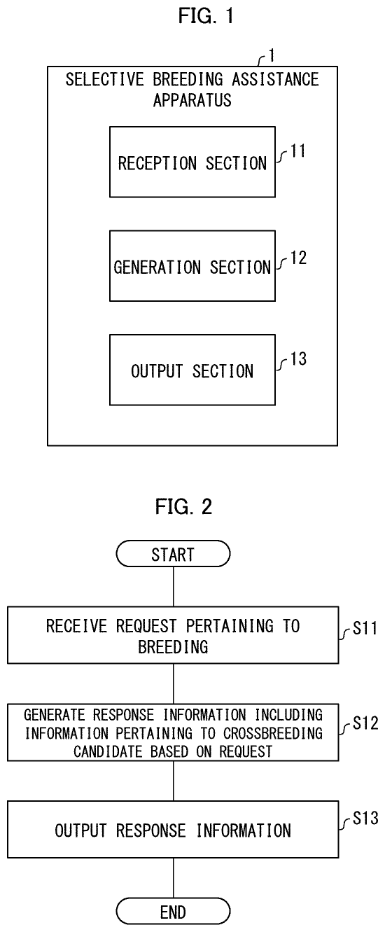

The following description will discuss a configuration of a selective breeding assistance apparatus 1 in accordance with the present example embodiment, with reference to FIG. 1. FIG. 1 is a block diagram illustrating the configuration of the selective breeding assistance apparatus 1. As illustrated in FIG. 1, the selective breeding assistance apparatus 1 includes a reception section (reception means) 11, a generation section (generation means) 12, and an output section (output means) 13.

The reception section 11 receives a request pertaining to breeding. The generation section 12 generates response information based on the request using a learned model which has learned a relation between a property of an existing variety and a breeding process of the existing variety, the response information including information pertaining to an existing variety that serves as a crossbreeding candidate for developing a new variety. The output section 13 outputs the response information.

Note that the term “property of an existing variety” indicates what property the existing variety has. For example, in addition to properties pertaining to a growing environment such as desiccation tolerance, high-temperature tolerance, and low-temperature tolerance, properties of harvests such as high yield (i.e., a large amount of harvest) and good taste, disease resistance, and the like are also included in the scope of the “property of an existing variety”. The term “breeding process of an existing variety” indicates how the existing variety has been developed. For example, parental varieties, the number of times of crossbreeding carried out until the existing variety is produced, a character obtained at the time of crossbreeding, a character lost at the time of crossbreeding, and the like are also included in the scope of the “breeding process of an existing variety”. Note that the term “character” means a morphological or physiological property.

According to the selective breeding assistance apparatus 1 having the above configuration, it is possible to generate useful response information including information pertaining to an existing variety that serves as a crossbreeding candidate for developing a new variety. Therefore, according to the configuration, it is possible to bring about an example advantage of suitably assisting breeding.

### (Selective Breeding Assistance Program)

The functions of the selective breeding assistance apparatus 1 described above can also be realized by a program. A selective breeding assistance program in accordance with the present example embodiment causes a computer to carry out: a process of receiving a request pertaining to breeding; a process of generating response information based on the request using a learned model which has learned a relation between a property of an existing variety and a breeding process of the existing variety, the response information including information pertaining to an existing variety that serves as a crossbreeding candidate for developing a new variety; and a process of outputting the response information. According to the selective breeding assistance program, it is possible to bring about an example advantage of suitably assisting breeding.

### (Selective Breeding Assistance Method)

The following description will discuss a selective breeding assistance method in accordance with the present example embodiment, with reference to FIG. 2. FIG. 2 is a flowchart illustrating a flow of the selective breeding assistance method in accordance with the first example embodiment of the present invention.

In S11, a computer receives a request pertaining to breeding. The request may be received via an arbitrary input apparatus. For example, the request may be received via a mouse, a keyboard, a touch panel, or an audio input apparatus.

In S12, the computer generates response information based on the request, which has been received in S11, using a learned model which has learned a relation between a property of an existing variety and a breeding process of the existing variety, the response information including information pertaining to an existing variety that serves as a crossbreeding candidate for developing a new variety.

In S13, the computer outputs the response information which has been generated in S12. The information may be output to an arbitrary apparatus. For example, the information may be output to a display apparatus so that the information is displayed, or the information may be output to an audio output apparatus so that the information is output as audio.

As described above, according to the selective breeding assistance method in accordance with the present example embodiment: the computer receives a request pertaining to breeding (S11); the computer generates response information based on the request, which has been received in S11, using a learned model which has learned a relation between a property of an existing variety and a breeding process of the existing variety, the response information including information pertaining to an existing variety that serves as a crossbreeding candidate for developing a new variety (S12); and the computer outputs the response information which has been generated in S12 (S13). According to the selective breeding assistance method, it is possible to bring about an example advantage of suitably assisting breeding.

An execution subject of each step in the selective breeding assistance method described above may be a single computer (e.g., the selective breeding assistance apparatus 1), or execution subjects of respective steps may be different computers. The same applies to a flow described later in the second and subsequent example embodiments.

### (Graph and Learning)

The following description will discuss a graph that is an example of information which can be used to assist selective breeding in the first example embodiment and in the subsequent example embodiments (hereinafter, referred to as “each example embodiment”). The following description will also discuss learning of the graph and prediction using the graph.

### (Graph)

The graph herein refers to data having a structure composed of a plurality of nodes and links connecting the nodes. A type of link representing a relation between nodes is also called a “relation”. Alternatively, the link may be called an “edge”. The graph roughly includes a directed graph in which each link has directionality, and an undirected graph in which each link has no directionality. It is possible to utilize either a directed graph or an undirected graph. It is also possible to use those graphs in combination.

In a case of using a graph in each example embodiment, a node in the graph only needs to represent a tangible or intangible element pertaining to breeding. For example, it is possible to utilize a graph including nodes representing various elements such as:
variety identification information (e.g., variety name, ID);
character of variety (e.g., disease resistance, heat tolerance, cold tolerance, high yielding ability, harvest taste, and the like);
genome information; and
classification as breeding material (e.g., a mid-mother plant which has a fault as a practical variety but has an excellent genetic characteristic, an introduction strain which is a strain obtained from a foreign country, a conventional variety which is a strain that has been bred in an old age and maintained, and the like).

Note that the graph may include a plurality of nodes corresponding to a single element. For example, since two varieties exist as crossbreeding parents of a certain variety, the crossbreeding parents are represented by two individual nodes. The same applies to other elements.

In a case where there are nodes corresponding to elements as described above, a link connecting such nodes represents:
a relation between a certain variety and another variety;
a relation between a certain element and a numerical value pertaining to the certain element;
a relation indicating that a certain element has a certain character; or the like.

For example, a link connecting a node indicating a certain variety to a node indicating another variety may represent a relation in which the certain variety is a crossbreeding parent of the another variety.

### (Learning and Prediction)

For a graph as described above, a machine learning technique can be applied to carry out graph-based relationship learning. Such learning makes it possible to carry out a classification process or a prediction process using a graph. In each example embodiment, such learning may be carried out as a part of selective breeding assistance, or a learned graph which has already been subjected to such learning may be used.

In graph-based relationship learning, first, a feature quantity of each node is calculated. The feature quantity may be in, for example, a vector form. By representing a feature quantity of each node by a feature quantity vector, it is possible to carry out learning also for a graph in which nodes of various forms mixedly exist. For example, graph-based relationship learning can also be carried out for a graph including an image, a numerical value, and the like indicating various elements as described above.

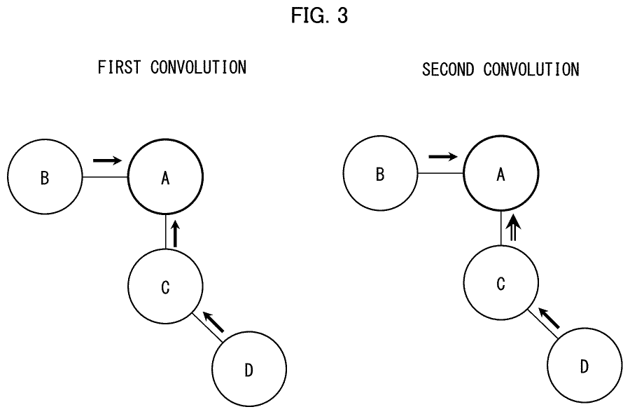

Next, the feature quantity of each node is updated based on a link connected to that node and a node which is a connection destination of the link. This process is similar to a convolution process in a convolutional neural network. This will be described with reference to FIG. 3. FIG. 3 is a diagram illustrating learning of feature quantities in graph-based relationship learning.

In a graph illustrated in FIG. 3, four nodes A through D are included. The nodes B and C are connected to the node A, and the node D is connected to the node C. After calculating initial feature quantities of these four nodes, a plurality of times of convolution are carried out as described below to update the feature quantities of the respective nodes.

In the first convolution, feature quantities of the nodes B and C connected to the node A are each multiplied by a predetermined weight and are then added to an initial feature quantity of the node A. For the node C, a feature quantity of the node D is multiplied by a predetermined weight and is then added to an initial feature quantity of the node C. Note that, in a case of an effective graph, the weight is adjusted according to a direction of the link.

In the second convolution, similarly to the first convolution, a feature quantity of a node linked to each node is multiplied by a predetermined weight, and is then added to a feature quantity of the each node. Here, the feature quantity of the node C reflects the feature quantity of the node D by the first convolution. Therefore, by the second convolution, not only the feature quantity of the node C but also the feature quantity of the node D are reflected in the node A.

By repeating the above-described process by a number of times corresponding to the hierarchy of nodes, feature quantities of nodes which are directly or indirectly connected to each other by links are mutually reflected. In graph-based relationship learning, a weight value used for the above-described weighting is optimized based on a known relationship between nodes. By using such a learned graph (which can also be called a learned model), it is possible to predict an inter-node relation and predict a link destination node, as described below.

### (Inter-Node Relation Prediction)

By carrying out the learning described above, it is possible to predict an inter-node relation which is not explicitly indicated in the original graph. In a case of carrying out inter-node relation prediction, a user may specify two nodes and make a request for returning a relation between those nodes. For example, in a case where a request of inquiring about a relation between a node of “variety A” and a node of “variety B” is input from a user, it is possible to predict, by inter-node relation prediction, whether or not a relation (i.e., a link) that connects these nodes is “good crossbreeding partner”. In the inter-node relation prediction, it is possible to calculate a probability (likelihood) of a prediction result. The same applies to node prediction described below.

### (Node Prediction)

By carrying out the above-described learning, it is also possible to predict a node that is connected to a certain node by a predetermined link. In a case of carrying out node prediction, a user may specify a single node and a link starting from that single node, and make a request for returning a link destination node. For example, it is assumed that a request for inquiring about a node connected to a node of “variety A” by a link of “character” is input from a user. In this case, for example, it is possible to predict, by node prediction, whether a node connected to the node of “variety A” by the link of “character” is “good taste” or “disease resistance”.

### Second Example Embodiment

### (Overview)

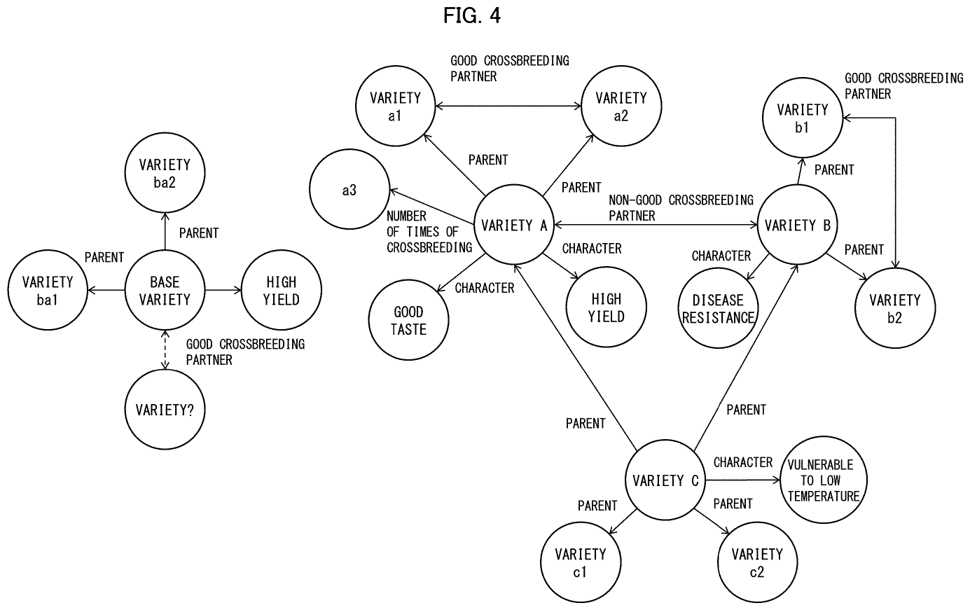

FIG. 4 is a diagram illustrating an overview of a selective breeding assistance method in accordance with the present example embodiment. In the present example embodiment, an example will be described in which a base variety graph and an existing variety graph are used to assist selective breeding.

The existing variety graph is a learned model and is a graph which includes a plurality of nodes pertaining to an existing variety and links indicating relationships between the plurality of nodes and which has learned the relationships between the plurality of nodes. The existing variety graph can also be called a knowledge graph. A set of nodes and links corresponding to a single existing variety may be referred to as an existing variety graph, or a set of nodes and links corresponding to a plurality of existing varieties may be collectively referred to as an existing variety graph.

For example, in FIG. 4, a graph including nodes of “variety A” through “variety C” is an existing variety graph. This existing variety graph includes nodes indicating that parents (which are crossbreeding parents; the same applies hereinafter) of the variety A, which is an existing variety, are a “variety a1” and a “variety a2”, nodes indicating that the variety A has characters of “high yield” and “good taste”, and a node indicating that “a3” times of crossbreeding have been taken to breed the variety A. The existing variety graph of FIG. 4 also indicates that parents of the “variety C” are the “variety A” and the “variety B”, and that the “variety A” and the “variety B” are “non-good crossbreeding partners”. As such, the existing variety graph illustrated in FIG. 4 has learned crossbreeding instances between existing varieties.

It is possible that links between varieties that are “good crossbreeding partners” are learned, and links between varieties that are “non-good crossbreeding partners” are not learned (i.e., such a case is regarded as a negative example). However, by learning a link between varieties that are “non-good crossbreeding partners”, there is an advantage that such learning is utilized to predict a positive example (in this example, a good crossbreeding partner) or such learning makes it possible to predict a risk.

A base variety graph is a graph including a plurality of nodes pertaining to a base variety, which is one of crossbreeding parents of a new variety to be bred. In FIG. 4, a graph including a node “base variety” is a base variety graph. The base variety graph includes nodes indicating that parents of the base variety are a “variety ba1” and a “variety ba2”, and a node indicating that the base variety is “high yield”. Such a base variety graph can be generated, for example, from various kinds of information which are recorded in a database in which pieces of information pertaining to breeding are accumulated.

For example, by receiving input of a base variety as a request from a user, it is possible to extract information indicating a variety that is a parent of the base variety, a character of the base variety, and the like from a database as described above, and generate a base variety graph. At this time, a request for a character or the like demanded of a new variety based on that base variety may also be received. Alternatively, a user may select, as the base variety, one of existing varieties indicated in the existing variety graph. In this case, an existing variety graph of the selected existing variety may be used as a base variety graph.

By training the existing variety graph as described above, it is possible to carry out link prediction for predicting which kinds of varieties are suitable for crossbreeding. That is, in the selective breeding assistance method in accordance with the present example embodiment, an existing variety to be crossbred with the base variety is predicted by link prediction, and response information corresponding to the predicted existing variety is generated and output.

For example, in the example of FIG. 4, it is possible to carry out link prediction for predicting which one of nodes indicating various varieties included in the existing variety graph seems to be connected to a node (more specifically, a node of “base variety”) included in the base variety graph by a link of “good crossbreeding partner”. Then, it is possible to generate and output response information indicating that the predicted variety is an existing variety to be crossbred with the base variety.

### (Apparatus Configuration)

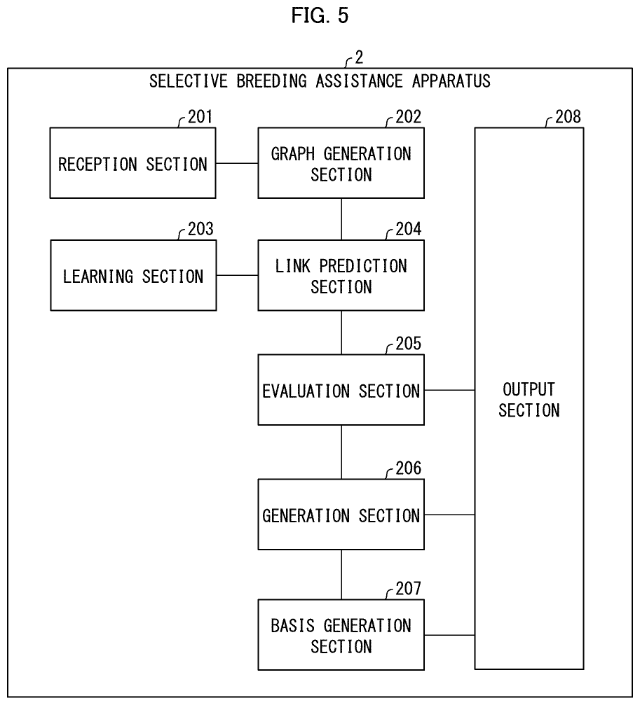

The following description will discuss a configuration of a selective breeding assistance apparatus 2 in accordance with the second example embodiment of the present invention, with reference to FIG. 5. FIG. 5 is a block diagram illustrating the configuration of the selective breeding assistance apparatus 2 in accordance with the present example embodiment.

As illustrated in FIG. 5, the selective breeding assistance apparatus 2 includes a reception section 201, a graph generation section 202, a learning section 203, a link prediction section 204, an evaluation section 205, a generation section 206, a basis generation section 207, and an output section 208.

In addition to these constituent elements, the selective breeding assistance apparatus 2 may include an input apparatus for receiving an input operation by a user, an output apparatus for outputting data output by the selective breeding assistance apparatus 2, a communication apparatus for enabling the selective breeding assistance apparatus 2 to communicate with another apparatus, and the like. An output mode of the output apparatus is arbitrary, and may be, for example, display output or audio output.

The reception section 201 receives a request pertaining to breeding. The request may include, for example, a character demanded of a new variety to be produced, information indicating a base variety that is a base of the new variety, and the like.

The graph generation section 202 generates, based on information pertaining to a base variety which is a base of a new variety to be developed by a user, a base variety graph representing that base variety in the form of graph. Specifically, the graph generation section 202 generates a base variety graph representing the base variety with a node indicating the base variety, nodes indicating parents of the base variety, a node indicating a character of the base variety, and edges representing relationships between the nodes. The information pertaining to the base variety may be included in a request received by the reception section 201 or may be acquired from a database in which pieces of information pertaining to breeding are accumulated, or the like.

The learning section 203 learns, based on various kinds of information pertaining to an existing variety, a relationship between nodes included in an existing variety graph, in other words, a relation between a property of the existing variety and a breeding process of the existing variety, and generates a learned existing variety graph. The existing variety graph can be considered to have learned crossbreeding instances between existing varieties. Unless otherwise specified, the existing variety graph refers to a graph which has been subjected to learning by the learning section 203. The learned existing variety graph may be read into the selective breeding assistance apparatus 2. In such a case, the learning section 203 may be omitted.

The link prediction section 204 predicts, by link prediction using a base variety graph and the foregoing existing variety graph, an existing variety to be crossbred with a base variety, the base variety graph including a plurality of nodes pertaining to the base variety which is one of crossbreeding parents of a new variety to be bred, and the link prediction being carried out for predicting a relationship between nodes which are not connected to each other by a link in a variety graph and the variety graph.

The evaluation section 205 evaluates, based on nodes included in an existing variety graph of the existing variety (i.e., a crossbreeding candidate) predicted by the link prediction section 204, suitability of the crossbreeding candidate as a crossbreeding partner for the base variety. The evaluation method will be described later.

The generation section 206 generates response information based on a request received by the reception section 201 and a learned model which has learned a relation between a property of an existing variety and a breeding process of the existing variety, the response information including information pertaining to an existing variety that serves as a crossbreeding candidate for developing a new variety. More specifically, the generation section 206 generates response information corresponding to an existing variety (in other words, a crossbreeding candidate) predicted by the link prediction section 204. As described above, the link prediction section 204 carries out link prediction using an existing variety graph. Therefore, the generation section 206 generates response information based on a result of link prediction by the link prediction section 204, and thereby generates response information based on a learned model.

The basis generation section 207 generates basis information indicating validity of response information generated by the generation section 206. The method for generating basis information will be described later.

The output section 208 outputs various pieces of information generated by the selective breeding assistance apparatus 2. For example, the output section 208 outputs response information generated by the generation section 206 and basis information indicated by the basis generation section 207. An output destination of the information is arbitrary. For example, in a case where the selective breeding assistance apparatus 2 includes an output apparatus as described above, the information may be output to the output apparatus. Alternatively, for example, the information may be output to an output apparatus provided outside the selective breeding assistance apparatus 2.

As described above, the learned model used by the selective breeding assistance apparatus 2 may be an existing variety graph that is a graph which includes a plurality of nodes pertaining to an existing variety and links indicating relationships between the plurality of nodes and which has learned the relationships between the plurality of nodes. According to the configuration, it is possible to generate and output proper response information pertaining to an existing variety that serves as a crossbreeding candidate, while taking into consideration mutual relationships between a crossbreeding result, parents, grandparents, a character, and the like of the existing variety.

As described above, the selective breeding assistance apparatus 2 may include the link prediction section 204 which predicts, by link prediction using a base variety graph and an existing variety graph that has learned crossbreeding instances between existing varieties, an existing variety to be crossbred with a base variety, the base variety graph including a plurality of nodes pertaining to the base variety which is one of crossbreeding parents of a new variety to be bred, and the link prediction being carried out for predicting a relationship between nodes which are not connected to each other by a link in the variety graph and the existing variety graph. The generation section 206 may generate response information corresponding to the existing variety predicted by the link prediction section 204. This makes it possible to recommend, to a user, an existing variety that seems to be proper as a crossbreeding candidate based on past crossbreeding instances.

### (Link Prediction)

As described above, the link prediction section 204 predicts, by link prediction using a base variety graph and an existing variety graph, an existing variety to be crossbred with a base variety. For example, the link prediction section 204 may predict, as in the example of FIG. 4, a node of an existing variety that is connected to a node of “base variety” by a link of “suitable crossbreeding partner”. The existing variety predicted by the link prediction section 204 serves as a crossbreeding candidate to be crossbred with the base variety.

The link prediction section 204 may predict, as an existing variety to be crossbred with the base variety, an existing variety that conforms to a specified condition. The specification of the condition may be carried out in advance or may be carried out by a user. In the latter case, the reception section 201 may receive input of a condition as a request.

For example, the link prediction section 204 may predict, as an existing variety to be crossbred with the base variety, an existing variety that satisfies at least one selected from the group consisting of a condition (1) that a variety which is obtained by crossbreeding the existing variety of interest with an existing variety having a predetermined character maintains the predetermined character and a condition (2) of having a character to be added to the base variety.

In a case where an existing variety satisfying the condition (1) is predicted as an existing variety to be crossbred with the base variety, it is possible to predict, as an existing variety to be crossbred with the base variety, a variety that is less likely to lose a predetermined character when being crossbred with the base variety. In a case where an existing variety satisfying the condition (2) is predicted as an existing variety to be crossbred with the base variety, it is possible to predict, as an existing variety to be crossbred with the base variety, a variety that is more likely to express a character to be added to the base variety when being crossbred with the base variety.

### (Evaluation of Crossbreeding Candidate)

As described above, the evaluation section 205 evaluates, based on nodes included in an existing variety graph of an existing variety (i.e., a crossbreeding candidate) predicted by the link prediction section 204, suitability of the crossbreeding candidate as a crossbreeding partner for the base variety. The following description will discuss evaluation by the evaluation section 205.

Each of nodes included in an existing variety graph of an existing variety which has been predicted to be crossbred with a base variety may indicate a factor that affects breeding of a new variety. For example, the following description assumes a case in which an existing variety graph includes nodes and links indicating the number of times of crossbreeding taken for generating an intended variety by crossbreeding using the existing variety as a parent. In this case, if the number of times of crossbreeding shown by those nodes and links is small, it is likely that, even if the existing variety is crossbred with the base variety, an intended new variety can be produced in a small number of times of crossbreeding.

In view of this, according to the configuration, suitability of the crossbreeding candidate as a crossbreeding partner for the base variety is evaluated based on nodes included in an existing variety graph of the existing variety predicted by the link prediction section 204. An evaluation method may be determined in advance based on target nodes or the like. The user may decide, according to the evaluation, whether to use the predicted existing variety as a crossbreeding partner for the base variety. According to the configuration, it is possible to contribute to appropriate selection of a variety to be crossbred with the base variety.

Various criteria for evaluation can be applied. For example, evaluation can be carried out on the basis of a degree of conformity to a request. For example, the following description assumes that a request includes a character demanded of a new variety. In this case, the evaluation section 205 may set evaluation of an existing variety corresponding to an existing variety graph which includes a node indicating that character to be higher than evaluation of an existing variety corresponding to an existing variety graph which does not include a node indicating that character.

The evaluation section 205 may express an evaluation result by a numerical value. In the present example embodiment, an example is described in which the evaluation section 205 calculates a recommendation level, which is a numerical value indicating suitability as a crossbreeding partner for the base variety. In this case, for example, by setting, as a rule, a relation between a node included in an existing variety graph of a crossbreeding candidate and a recommendation level in advance, the evaluation section 205 can calculate a recommendation level of each crossbreeding candidate according to the rule.

### (Method of Generating Basis Information)

As described above, the basis generation section 207 generates basis information indicating validity of response information generated by the generation section 206. For example, in a case where a request received by the reception section 201 includes information indicating a character that is demanded of a new variety, the basis generation section 207 may generate basis information including at least one selected from the group consisting of information pertaining to breeding of an existing variety that serves as a crossbreeding candidate and information pertaining to a character of the existing variety that serves as a crossbreeding candidate. This configuration allows a user to refer to response information while taking into consideration basis information, and to precisely determine validity of the response information.

Examples of information pertaining to breeding of an existing variety that serves as a crossbreeding candidate include parents of the existing variety that serves as a crossbreeding candidate, characters of the parents, a character deteriorated or added in crossbreeding until the existing variety is produced, the number of times of crossbreeding, and the like. Examples of information pertaining to a character of a variety that serves as a crossbreeding candidate include a character that is possessed by the variety which serves as a crossbreeding candidate, a character that is not possessed by the variety which serves as a crossbreeding candidate, and the like.

Various techniques can be applied as a method of generating basis information. For example, it is assumed that information indicating a character that is demanded of a new variety is included in a request received by the reception section 201. In this case, the basis generation section 207 may check for the presence or absence of the character in a crossbreeding candidate and parents thereof and, if it has been confirmed that there is the character, the basis generation section 207 may generate basis information indicating a variety having the character. In a case where the presence of the character is not confirmed even in the parental generation, the basis generation section 207 may search back through strains until the character is confirmed. Such confirmation of a character by tracing strains can be easily carried out by using an existing variety graph.

(Basis generation with respect to link prediction result)

The basis generation section 207 can generate basis information also by analyzing a base variety graph and an existing variety graph. The following description will discuss a method of generating basis information by analyzing a base variety graph and an existing variety graph.

For example, the basis generation section 207 may mine one or more rules from the base variety graph and the existing variety graph using principal component analysis (PCA) reliability based on open-world assumption (OWA). The basis generation section 207 may generate basis information using one or more rules that have been mined. For example, a method described in the following literature can be applied to mining of a rule.
Luis Galarraga et. al, “Fast rule mining in ontological knowledge bases with AMIE +”, The VLDB Journal (2015) 24:707-730

For example, a rule to be processed by the basis generation section 207 is expressed by

B₁ΛB₂Λ . . . Bₙ⇒r(x,y)

using Head r(x, y) and Body {B1, . . . , Bn}. This rule may also be expressed as

\vec{B}⇒r(x,y)

using a vector representation. Here, Head r(x, y) is also called an atom.

As a condition of the mining process, the basis generation section 207 uses the following conditions to carry out the mining process:
Connected: all values (variables, entities) in a rule are shared between different atoms;
Closed: all variables in a rule appear two times or more; and
Not reflexive: a rule containing a reflective atom (such as r(x, x)) is not mined.

The basis generation section 207 may use a head coverage (hc) defined by

$$hc ( \vec{B} ⇒ r ( x , y ) ) := \frac{supp ( \vec{B} ⇒ r ( x , y ) )}{size ( r )}$$

and PCA reliability defined by

$${conf}_{\text{?}} ( \vec{B} ⇒ r ( x , y ) ) := \frac{supp ( \vec{B} ⇒ r ( x , y ) )}{\# ( x , y ) : {\text{?}}_{z \text{?}} , … , {z}_{m} \text{?} {y}^{′} : \vec{B} \text{?} r ( x , {y}^{′} )}$$

$$\text{?} \text{indicates text missing or illegible when filed}$$

to carry out the mining process. By using PCA reliability, it is possible to mine a rule with higher accuracy, as compared with a case of using standard reliability. Therefore, by using the above configuration, it is possible for the basis generation section 207 to generate highly reliable basis information.

For example, it is assumed that the basis generation section 207 has mined a rule “crossbreeding result is good” when crossbreeding a variety which satisfies a condition “desirable character has not been lost in parental generation of variety to be crossbred”. In this case, if the link prediction section 204 has predicted an existing variety to be crossbred with the base variety, the basis generation section 207 may generate, as a basis of the prediction, basis information indicating that “desirable character has not been lost in parental generation of variety to be crossbred”.

### (Flow of Process)

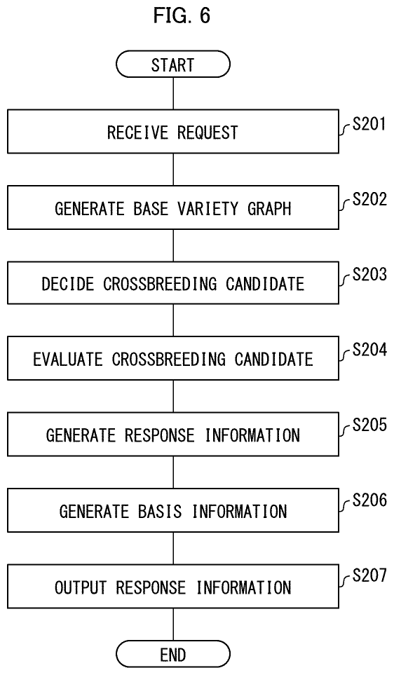

The following description will discuss a flow of a process (selective breeding assistance method) carried out by the selective breeding assistance apparatus 2, with reference to FIG. 6. FIG. 6 is a flowchart illustrating the flow of the process carried out by the selective breeding assistance apparatus 2.

In S201, the reception section 201 receives a request pertaining to breeding. In S201, for example, a request is received which indicates a character demanded of a new variety to be produced, a base variety, and the like. Subsequently, in S202, the graph generation section 202 generates a base variety graph based on the information input in S201.

In S203, the link prediction section 204 predicts, by link prediction using the base variety graph generated in S202 and an existing variety graph, an existing variety to be crossbred with the base variety, and decides the existing variety as a crossbreeding candidate. The link prediction section 204 may decide a plurality of crossbreeding candidates. In this case, the basis generation section 207 may generate basis information indicating a basis of a prediction result by the link prediction section 204 by analyzing the base variety graph and the existing variety graph.

In S204, the evaluation section 205 evaluates, based on nodes included in an existing variety graph of the crossbreeding candidate decided in S203, suitability as a crossbreeding partner for a base variety of the crossbreeding candidate. For example, the evaluation section 205 may calculate, from nodes included in the existing variety graph of the crossbreeding candidate, a recommendation level indicating suitability of the crossbreeding candidate as a crossbreeding partner for the base variety. In a case where a plurality of crossbreeding candidates have been decided in S203, the evaluation section 205 evaluates each of the decided crossbreeding candidates.

In S205, the generation section 206 generates response information based on the crossbreeding candidate decided in S203 and the request received in S201. As described above, the existing variety graph is a learned model which has learned a relation between a property of an existing variety and a breeding process of the existing variety. Then, a crossbreeding candidate is decided by link prediction using the existing variety graph. Therefore, it is possible to say that, in S205, response information is generated based on a request received in S201 and a learned model which has learned a relation between a property of an existing variety and a breeding process of the existing variety.

For example, the generation section 206 may generate response information indicating a crossbreeding candidate of which an evaluation result in S204 is up to a predetermined ordinal number among the crossbreeding candidates decided in S203. For example, the generation section 206 may generate response information indicating, among the crossbreeding candidates decided in S203, a crossbreeding candidate that conforms to the request received in S201. Besides the above examples, for example, the generation section 206 may generate response information indicating the crossbreeding candidate decided in S203 and the evaluation result in S204.

In S206, the basis generation section 207 generates basis information indicating validity of the response information generated in S205. For example, the basis generation section 207 may refer to an existing variety graph, identify at least one selected from the group consisting of information pertaining to breeding of a crossbreeding candidate and information pertaining to a character of the crossbreeding candidate, and generate basis information indicating the identified information.

In S207, the output section 208 outputs the response information which has been generated in S206. In this case, the output section 208 may output the basis information which has been generated in S206. Thus, the process of FIG. 6 ends.

### Example of Response Information

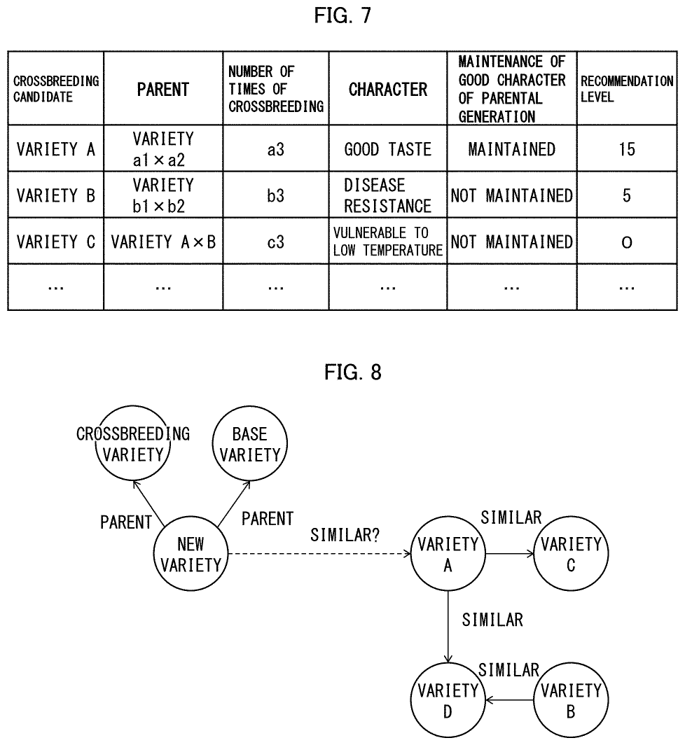

In S207, response information as illustrated in FIG. 7 for example may be output. FIG. 7 is a diagram illustrating an example of response information. The response information illustrated in FIG. 7 includes six items in total, i.e., “crossbreeding candidate”, “parents”, “number of times of crossbreeding”, “character”, “maintenance of good character of parental generation”, and “recommendation level”.

The “crossbreeding candidate” is predicted by the link prediction section 204. In the example of FIG. 7, varieties A through C are included in the crossbreeding candidates. The “parents” indicate parent varieties of a crossbreeding candidate of interest. The “number of times of crossbreeding” indicates the number of times of crossbreeding taken for producing a crossbreeding candidate of interest. The “character” indicates a character of a crossbreeding candidate of interest. The “maintenance of good character of parental generation” indicates whether or not a crossbreeding candidate of interest maintains a good character possessed by a parental generation thereof (Yes: maintained, No: not maintained). The “recommendation level” indicates an evaluation result by the evaluation section 205 with respect to a crossbreeding candidate of interest.

Among those items, “parents”, “number of times of crossbreeding”, “character”, and “maintenance of good character of parental generation” can be identified from an existing variety graph. The basis generation section 207 may generate such information as basis information.

The “recommendation level” may be calculated by the evaluation section 205 based on various kinds of information identified from an existing variety graph. For example, in a case of evaluation on the basis of a degree of conformity to a request, the evaluation section 205 may calculate the recommendation level so that a recommendation level of a crossbreeding candidate corresponding to an existing variety graph including a node indicating the requested character is higher than a recommendation level of a crossbreeding candidate corresponding to an existing variety graph that does not include a node indicating the character. The evaluation section 205 may calculate the recommendation level while taking into consideration a crossbreeding candidate, characters of parents of the crossbreeding candidate, the number of times of crossbreeding, and the like that are indicated in an existing variety graph.

In the example of FIG. 7, recommendation levels of varieties A through C are 15, 5, and 0, respectively. For example, in a case where a rule as described below has been set in advance, the evaluation section 205 can calculate a recommendation level of each crossbreeding candidate according to the rule. Such a rule may be as follows: if the number of times of crossbreeding is equal to or less than a threshold, the recommendation level is +5; if having one of requested characters, the recommendation level is +5; and if a good character of parental generation is maintained, the recommendation level is +5.

### Third Example Embodiment

### (Overview)

FIG. 8 is a diagram illustrating an overview of a selective breeding assistance method in accordance with the present example embodiment. In the present example embodiment, an example will be described in which a new variety graph including a plurality of nodes pertaining to a new variety to be bred and a plurality of existing variety graphs generated for a respective plurality of existing varieties are used to assist selective breeding.

In the selective breeding assistance method in accordance with the present example embodiment, information for identifying a new variety to be produced is received as a request pertaining to breeding. For example, parents of a new variety to be produced, that is, a base variety and a crossbreeding variety to be crossbred with the base variety may be received as a request.

Next, in the selective breeding assistance method in accordance with the present example embodiment, a new variety graph is generated based on the request. In the example of FIG. 8, a new variety graph indicates that nodes of “crossbreeding variety” and “base variety” are connected to a node “new variety” by links “parent”.

In the selective breeding assistance method in accordance with the present example embodiment, an existing variety having a predetermined relationship with the new variety is identified by link prediction using the new variety graph generated as described above and the existing variety graphs generated for the respective plurality of existing varieties. The existing variety graphs to be used are generated for the respective plurality of existing varieties, and have learned the predetermined relationship between the plurality of existing varieties.

In the example of FIG. 8, an existing variety (hereinafter, referred to also as a similar variety) that is similar to the new variety is predicted by link prediction using an existing variety graph which has learned similarity to the existing varieties A through D. In this example, the learning is carried out in such a manner that dissimilar varieties are not connected by a link “similar” (i.e., dissimilarity is regarded as a negative example). However, a link “dissimilar” may be learned. The existing varieties A through D include nodes and links indicating parents, characters, and the like thereof, but such nodes and links are not illustrated in FIG. 8.

Information indicating what kind of variety is a similar variety of a new variety is information that pertains to an existing variety serving as a crossbreeding candidate for developing a new variety, and that is useful for developing the new variety. Therefore, by generating and outputting response information indicating an existing variety identified as described above, it is possible to suitably assist breeding of a new variety.

It is also possible to carry out evaluation of a similar variety identified as described above, and decide a crossbreeding candidate according to the evaluation result. For example, the following description assumes a case of receiving a request indicating that a character of a new variety to be produced is “disease resistance”. In this case, if the identified similar variety has the character “disease resistance”, response information may be generated which recommends, as a crossbreeding candidate for developing a new variety, a crossbreeding variety included in a new variety graph. In contrast, if the identified similar variety does not have the character “disease resistance”, response information may be generated which indicates that a crossbreeding variety included in a new variety graph is not suitable as a crossbreeding candidate for developing a new variety. In this case, the user may make a request specifying a different crossbreeding variety.

### (Apparatus Configuration)

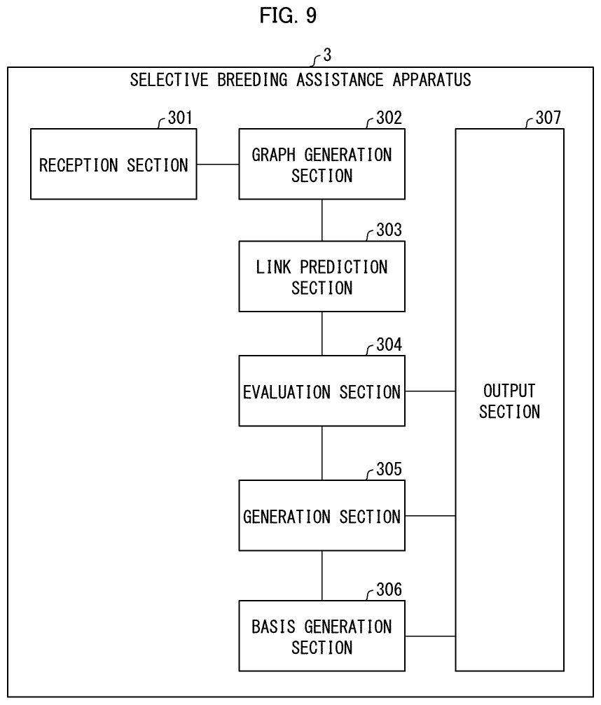

The following description will discuss a configuration of a selective breeding assistance apparatus 3 in accordance with the third example embodiment of the present invention, with reference to FIG. 9. FIG. 9 is a block diagram illustrating the configuration of the selective breeding assistance apparatus 3 in accordance with the present example embodiment.

As illustrated in FIG. 9, the selective breeding assistance apparatus 3 includes a reception section 301, a graph generation section 302, a link prediction section 303, an evaluation section 304, a generation section 305, a basis generation section 306, and an output section 307. As with the selective breeding assistance apparatus 2 in accordance with the second example embodiment, the selective breeding assistance apparatus 3 may include, in addition to those constituent elements, a learning section, an input apparatus, an output apparatus, a communication apparatus, and the like.

The reception section 301 receives a request pertaining to breeding. This request includes information for identifying a new variety to be produced. Specifically, the request includes information indicating a base variety which is a base of the new variety and a crossbreeding variety which is to be crossbred with the base variety. The request may include information indicating a character that is demanded of a new variety to be produced and a user demand pertaining to breeding such as an upper limit value of the number of times of crossbreeding.

The graph generation section 302 generates a new variety graph based on the request. For example, the graph generation section 302 may generate a new variety graph (see FIG. 8) in which a node indicating a new variety is connected to a node indicating a base variety and a node indicating a crossbreeding variety by links indicating that the nodes are parents of the new variety. The new variety graph may include nodes and links indicating parents, characters, and the like of the base variety and the crossbreeding variety.

The link prediction section 303 identifies an existing variety that has a predetermined relationship with a new variety by link prediction using a new variety graph including a plurality of nodes pertaining to the new variety to be bred and existing variety graphs generated for a plurality of existing varieties. The predetermined relationship may be a relationship of being similar as in the example of FIG. 8, or may be another relationship. For example, the link prediction section 303 may identify an existing variety that is not similar to a new variety, or can identify an existing variety that belongs to the same classification as a new variety, an existing variety that has commonality in character with a new variety, or the like.

The evaluation section 304 evaluates, based on nodes included in an existing variety graph of the existing variety identified by the link prediction section 303, i.e., an existing variety having a predetermined relationship with the new variety, suitability of a crossbreeding variety included in the new variety graph as a crossbreeding partner for a base variety. For example, in a case where the link prediction section 303 has identified a similar variety, the evaluation section 304 may evaluate the similar variety as having suitability if a node indicating a requested character is included in an existing variety graph of the similar variety, and may evaluate the similar variety as having no suitability if such a node is not included in the existing variety graph of the similar variety.

The generation section 305 generates response information based on a request received by the reception section 301 and a learned model which has learned a relation between a property of an existing variety and a breeding process of the existing variety, response information including information pertaining to an existing variety that serves as a crossbreeding candidate for developing a new variety. More specifically, the generation section 305 generates response information pertaining to the existing variety that has been identified by the link prediction section 303. As described above, the link prediction section 303 carries out link prediction using an existing variety graph, which is a learned model. Therefore, the generation section 305 generates response information based on a result of link prediction by the link prediction section 303, and thereby generates response information based on the learned model.

The generation section 305 may determine whether or not a crossbreeding variety s suitable based on an evaluation result of the evaluation section 304 and generate response information in accordance with the determination result. For example, it is assumed that a character of a new variety to be produced is requested, and the link prediction section 303 has identified a similar variety of the new variety. In this case, the evaluation section 304 may evaluate the identified similar variety based on whether or not the identified similar variety has the requested character. In a case where the evaluation section 304 has evaluated that the identified similar variety has the requested character, the generation section 305 may generate response information for recommending, as a crossbreeding candidate for developing the new variety, a crossbreeding variety included in the new variety graph. In contrast, in a case where the evaluation section 304 has evaluated that the identified similar variety does not have the requested character, the generation section 305 may generate response information indicating that a crossbreeding variety included in the new variety graph is not suitable as a crossbreeding candidate for developing the new variety.

The basis generation section 306 generates basis information indicating validity of response information generated by the generation section 305. Specifically, the basis generation section 306 generates basis information including at least one selected from the group consisting of information pertaining to breeding of the existing variety that serves as a crossbreeding candidate and information pertaining to a character of the existing variety that serves as a crossbreeding candidate. For example, the basis generation section 306 may generate basis information indicating parents, the number of times of crossbreeding, a character, and the like of the existing variety that has been identified by the link prediction section 303. Here, such properties of the existing variety are those identified based on nodes and links included in an existing variety graph of the existing variety. The basis generation section 306 may generate basis information pertaining to a result of link prediction by the link prediction section 303 by analyzing the new variety graph and the existing variety graph.

The output section 307 outputs, for example, response information generated by the generation section 305. An output destination of the information is not particularly limited, as with the output section 208 in accordance with the second example embodiment.

As described above, the selective breeding assistance apparatus 3 includes the link prediction section 303 that identifies an existing variety that has a predetermined relationship with a new variety by link prediction using a new variety graph including a plurality of nodes pertaining to the new variety to be bred and existing variety graphs generated for a plurality of existing varieties. Then, the generation section 305 generates response information pertaining to the existing variety that has been identified by the link prediction section 303. Information pertaining to an existing variety having a predetermined relationship with a new variety to be bred is useful in breeding of the new variety. Therefore, according to the configuration, it is possible to provide information useful for breeding of a new variety.

### (Flow of Process)

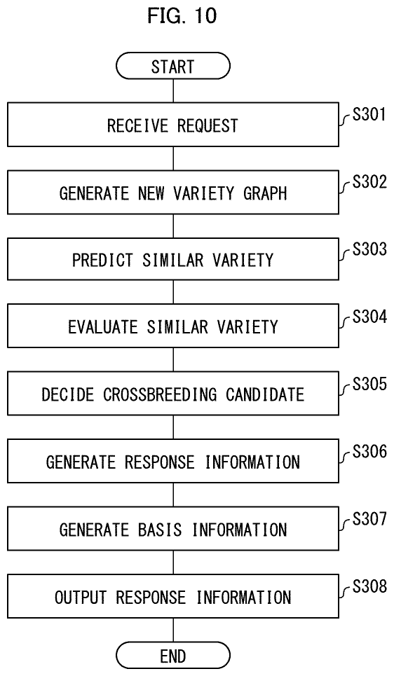

The following description will discuss a flow of a process (selective breeding assistance method) carried out by the selective breeding assistance apparatus 3, with reference to FIG. 10. FIG. 10 is a flowchart illustrating the flow of the process carried out by the selective breeding assistance apparatus 3.

In S301, the reception section 301 receives a request pertaining to breeding. In S301, for example, a request is received which indicates a character demanded of a new variety to be produced, a base variety which is a base of the new variety, a crossbreeding variety to be crossbred with the base variety, and the like. Subsequently, in S302, the graph generation section 302 generates a new variety graph based on the information input in S301.

In S303, the link prediction section 303 predicts, by link prediction using the new variety graph generated in S302 and existing variety graphs, a similar variety that is an existing variety similar to the new variety. In association with the process of S303, the basis generation section 306 may analyze the new variety graph and the existing variety graphs to generate basis information indicating a basis of the prediction result by the link prediction section 303.

In S304, the evaluation section 304 evaluates, based on nodes included in an existing variety graph of the similar variety predicted in S303, suitability of a crossbreeding variety included in the new variety graph as a crossbreeding partner for a base variety. For example, the evaluation section 304 may evaluate the crossbreeding variety as having suitability if a node indicating a requested character is included in an existing variety graph of the similar variety, and may evaluate the crossbreeding variety as having no suitability if such a node is not included in the existing variety graph of the similar variety.

In S305, the generation section 305 decides a crossbreeding candidate based on the variety predicted in S303, and in subsequent S306, the generation section 305 generates response information indicating the crossbreeding candidate decided in S305. As described above, the new variety graph is generated based on the request, the existing variety graphs are each a learned model, and the similar variety is identified by link prediction using the existing variety graphs. Therefore, it is possible to say that, in S306, response information is generated based on the request and the learned model.

For example, in a case of being evaluated as having suitability in S304, the generation section 305 may decide, in S305, a crossbreeding variety included in the new variety graph as a crossbreeding candidate for developing the new variety, and the generation section 305 may generate, in S306, response information indicating this crossbreeding candidate. In contrast, in a case of being evaluated as having no suitability in S304, the generation section 305 may not decide a crossbreeding candidate in S305, and the generation section 305 may generate, in S306, response information indicating that a crossbreeding variety included in the new variety graph is not suitable as a crossbreeding candidate for developing the new variety.

The process of S305 may be omitted. In this case, the generation section 305 may generate response information indicating the similar variety predicted in S303 and the evaluation result in S304.

In S307, the basis generation section 305 generates basis information indicating validity of the response information generated in S306. For example, the basis generation section 306 may generate basis information indicating parents, the number of times of crossbreeding, a character, or the like of the similar variety predicted in S303.

In S308, the output section 307 outputs the response information which has been generated in S306. In this case, the output section 307 may output the basis information which has been generated in S307. Thus, the process of FIG. ends.

### (Identification of Similar Variety of Base Variety)

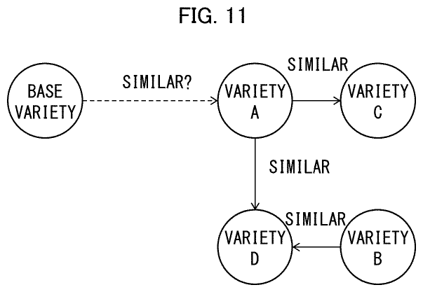

The link prediction section 303 may identify a similar variety of a base variety, instead of identifying a similar variety of a new variety. This will be described with reference to FIG. 11. FIG. 11 is a diagram illustrating an overview of a selective breeding assistance method including a process of identifying a similar variety of a base variety.

In the example of FIG. 11, link prediction for predicting an existing variety (hereinafter, referred to as a similar variety of the base variety) similar to the base variety is carried out using a base variety graph and existing variety graphs of existing varieties A through D. The base variety graph is similar to that described in the second example embodiment, and the existing variety graphs are similar to those in the example of FIG. 8.

The existing variety graphs each indicate, for example, in what kind of crossbreeding the similar variety of the base variety has been produced, what kind of variety has been crossbred with the similar variety of the base variety, and what kind of variety has been produced by the crossbreeding. Therefore, it is possible to suitably assist breeding by generating and outputting response information including information pertaining to the similar variety of the base variety. For example, response information may be generated which indicates information indicating a similar variety of the base variety, a variety that has been crossbred with a similar variety of the base variety, a variety whose parent is a similar variety of the base variety, a character of a variety whose parent is a similar variety of the base variety, or the like.

Note that an existing variety having a relationship other than being similar to the base variety may be identified. For example, it is possible to identify an existing variety that is not similar to the base variety, or identify an existing variety that belongs to the same classification as the base variety, an existing variety that has commonality in character with the base variety, or the like.

It is also possible to carry out evaluation of a similar variety of the base variety identified as described above, and decide a crossbreeding candidate according to the evaluation result. For example, the following description assumes a case of receiving a request indicating that a character of a new variety to be produced is “disease resistance”. In this case, if an offspring variety obtained by crossbreeding a similar variety of the base variety and a certain existing variety has a character “disease resistance”, response information may be generated which recommends the certain existing variety as a crossbreeding candidate for developing the new variety.

### (Flow of Process)

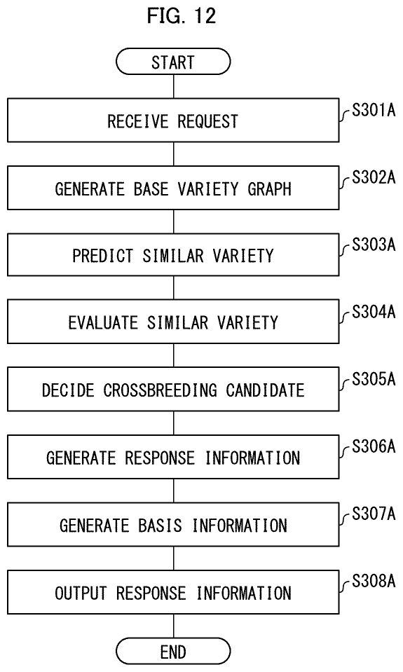

The following description will discuss a flow of a process (selective breeding assistance method) carried out by the selective breeding assistance apparatus 3 when identifying a similar variety of a base variety, with reference to FIG. 12. FIG. 12 is a flowchart illustrating the flow of the process carried out by the selective breeding assistance apparatus 3 when identifying a similar variety of a base variety.

In S301A, the reception section 301 receives a request pertaining to breeding. In S301A, for example, a request is received which indicates a character demanded of a new variety to be produced, a base variety which is a base of the new variety, and the like. Subsequently, in S302A, the graph generation section 302 generates a base variety graph based on the information input in S301A.

In S303A, the link prediction section 303 predicts a similar variety of the base variety by link prediction using the base variety graph generated in S302A and existing variety graphs. The link prediction section 303 may predict a plurality of similar varieties of the base variety. In association with the process of S303A, the basis generation section 306 may analyze the base variety graph and the existing variety graphs to generate basis information indicating a basis of the prediction result by the link prediction section 303.

In S304A, the evaluation section 304 evaluates the similar variety of the base variety predicted in S303A based on an existing variety graph of the similar variety. For example, the evaluation section 304 may carry out evaluation based on a degree to which an offspring generation variety of the similar variety conforms to the request. For example, the evaluation section 304 may evaluate an offspring generation variety which has the requested character higher than an offspring generation variety which does not have the requested character. In a case where a plurality of similar varieties are predicted in S303A, the evaluation section 304 carries out evaluation for each of the decided similar varieties. The evaluation section 304 may carry out evaluation while taking into consideration a generation (grandchild generation and subsequent generations) below the offspring generation of the similar variety.

In S305A, the generation section 305 decides a crossbreeding candidate based on the similar variety of the base variety predicted in S303A, and in subsequent S306A, the generation section 305 generates response information indicating the crossbreeding candidate decided in S305A. As described above, the base variety graph is generated based on the request, the existing variety graphs are each a learned model, and the similar variety is identified by link prediction using the existing variety graphs. Therefore, it is possible to say that, in S306A, response information is generated based on the request and the learned model.

For example, the generation section 305 may generate response information indicating a similar variety of which an evaluation result in S304A is up to a predetermined ordinal number among similar varieties predicted in S303A.

Alternatively, for example, the generation section 305 may generate response information indicating a similar variety that derives an offspring generation in which a character conforming to the request received in S301A appears, among similar varieties predicted in S303A. The generation section 305 may generate response information indicating, in addition to or in place of a similar variety of the base variety, a variety that has been crossbred with the similar variety of the base variety and that derives an offspring generation in which a character conforming to the request appears.

In S307A, the basis generation section 306 generates basis information indicating validity of the response information generated in S306A. For example, the basis generation section 306 may refer to an existing variety graph, identify at least one selected from the group consisting of information pertaining to breeding of the crossbreeding candidate decided in S305 and information pertaining to a character of the crossbreeding candidate, and generate basis information indicating the identified information.

In S308A, the output section 307 outputs the response information which has been generated in S306A. In this case, the output section 307 may output the basis information which has been generated in S307A. Thus, the process of FIG. 12 ends.

As described above, the link prediction section 303 may identify an existing variety that has a predetermined relationship with the base variety by link prediction using the base variety graph and the existing variety graphs. Then, the generation section 305 may generate response information pertaining to the existing variety that has been identified by the link prediction section 303. Information pertaining to an existing variety having a predetermined relationship with the base variety, which is one of crossbreeding parents of the new variety, is useful in breeding of the new variety. Therefore, according to the configuration, it is possible to provide information useful for breeding using the base variety.

### Fourth Example Embodiment

### (Overview)

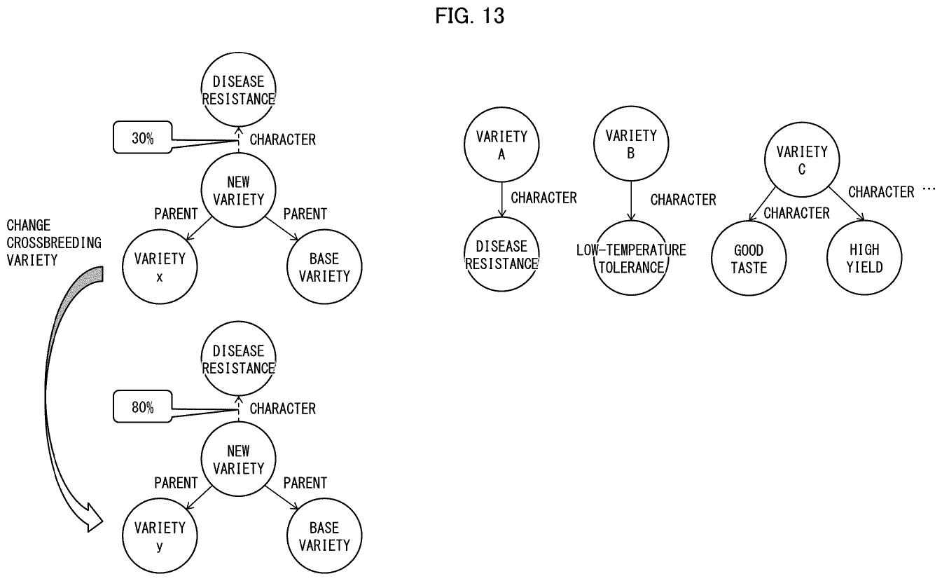

FIG. 13 is a diagram illustrating an overview of a selective breeding assistance method in accordance with the present example embodiment. In the present example embodiment, an example will be described in which a crossbreeding candidate of a new variety that conforms to a request is searched while updating a new variety graph including a plurality of nodes pertaining to the new variety to be bred.

In the present example embodiment, as in the third example embodiment, link prediction is carried out using a new variety graph and existing variety graphs. A new variety graph indicated in an upper left part of FIG. 13 includes nodes and links indicating that parents of the new variety is a “variety x” and a “base variety”.

Existing variety graphs of existing varieties A through C illustrated in FIG. 13 include nodes and links indicating that the existing variety A has a character “disease resistance”, that the existing variety B has a character “low-temperature tolerance”, and that the existing variety C has characters “good taste” and “high yield”. Note that other nodes and links are not illustrated.

By training the existing variety graphs for various existing varieties as described above, it is possible to carry out link prediction for predicting what variety is likely to have what character. That is, in the selective breeding assistance method in accordance with the present example embodiment, a tentative new variety graph is generated, and a probability that a new variety indicated in the tentative new variety graph has the requested character is predicted by link prediction.

For example, in the example of FIG. 13, it is predicted, in the upper left new variety graph, that a probability that the node of “disease resistance” will be connected to the node of “new variety” by the link of “character” is 30%. This probability is not high enough.

Therefore, as illustrated in the lower left part of FIG. 13, a node connected to the node of “new variety” by the link of “parent” in the new variety graph is changed from “variety x” to “variety y”, and link prediction is carried out again. Thus, a prediction result of the probability that the node of “disease resistance” will be connected to the node of “new variety” by the link of “character” is changed to 80%.

According to the selective breeding assistance method in accordance with the present example embodiment, it is possible to recommend the “variety y” as a crossbreeding candidate for imparting the character of disease resistance to a new variety, based on the result of the above-described process.

### (Apparatus Configuration)

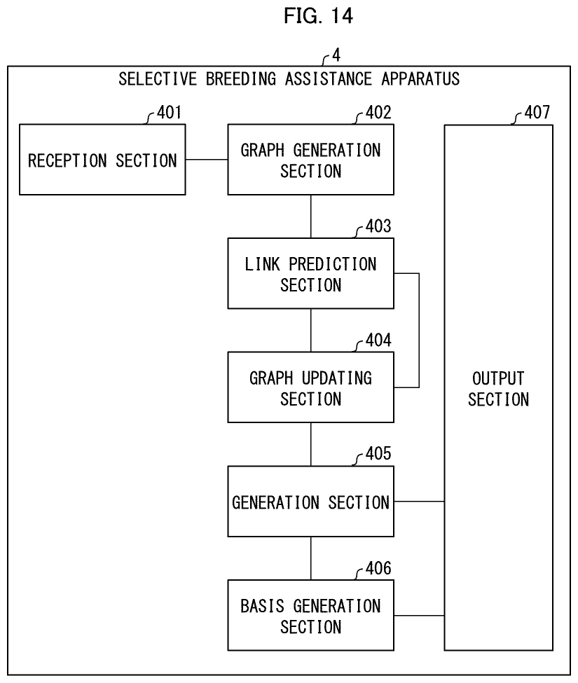

The following description will discuss a configuration of a selective breeding assistance apparatus 4 in accordance with the fourth example embodiment of the present invention, with reference to FIG. 14. FIG. 14 is a block diagram illustrating the configuration of the selective breeding assistance apparatus 4 in accordance with the present example embodiment.

As illustrated in FIG. 14, the selective breeding assistance apparatus 4 includes a reception section 401, a graph generation section 402, a link prediction section 403, a graph updating section 404, a generation section 405, a basis generation section 406, and an output section 407. As with the selective breeding assistance apparatus 2 in accordance with the second example embodiment, the selective breeding assistance apparatus 4 may include, in addition to those constituent elements, a learning section, an input apparatus, an output apparatus, a communication apparatus, and the like.

The reception section 401 receives a request pertaining to breeding. This request includes information for identifying a new variety to be produced. For example, the request may include information indicating a base variety which is a base of the new variety and a crossbreeding variety which is to be crossbred with the base variety. The request may include information indicating a character that is demanded of a new variety to be produced and information indicating a user demand pertaining to breeding such as an upper limit value of the number of times of crossbreeding.

The graph generation section 402 generates a new variety graph based on the request. For example, the graph generation section 402 may generate a new variety graph (see FIG. 13) in which a node indicating a new variety is connected to a node indicating a base variety and a node indicating a crossbreeding variety by links indicating that the nodes are parents of the new variety. The new variety graph may include nodes and links indicating parents, characters, and the like of the base variety and the crossbreeding variety.

The link prediction section 403 calculates, by link prediction using the new variety graph generated by the graph generation section 402 and a learned existing variety graph, a probability that a node indicating a predetermined character links to a node included in the new variety graph. The predetermined character is identified based on a request. For example, in a case where a character demanded of a new variety to be produced is requested, the link prediction section 403 calculates a probability that a node indicating the character links to a node (e.g., a node of “new variety” in the example of FIG. 13) included in the new variety graph.

The graph updating section 404 updates the new variety graph. Specifically, the graph updating section 404 carries out a process of replacing a node indicating a parent of the new variety included in the new variety graph with a node of another variety.

The updating of the new variety graph may be carried out according to input by a user or may be carried out automatically. In the former case, the graph updating section 404 may cause the output section 407 to output a list of existing varieties extracted from the existing variety graphs so that the user selects a new crossbreeding candidate among those existing varieties. In the latter case, the graph updating section 404 may select a new crossbreeding candidate from among existing varieties extracted from the existing variety graphs. A new crossbreeding candidate may be selected from existing varieties that derive offspring generations in which individuals having a requested character are present.

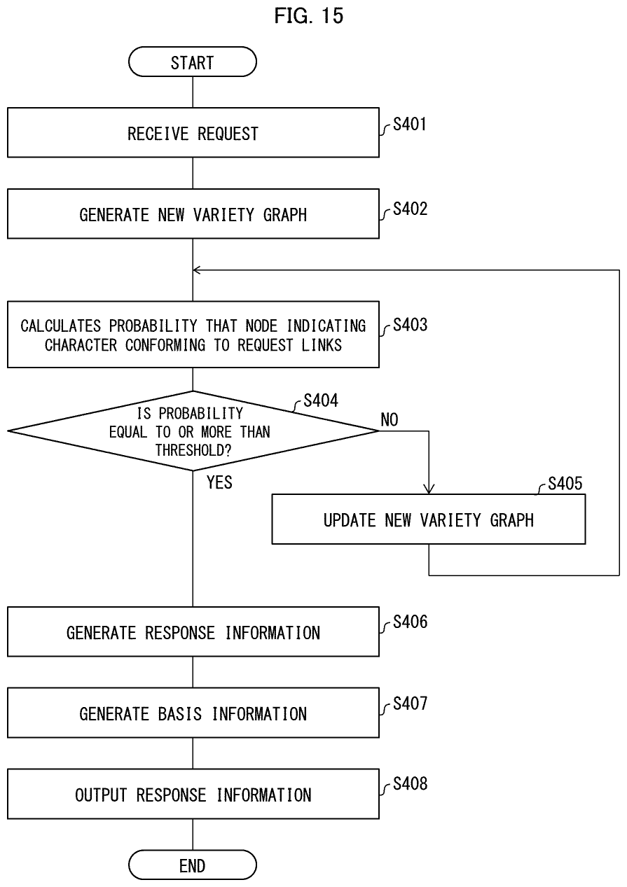

The generation section 405 generates response information based on a request received by the reception section 401 and a learned model which has learned a relation between a property of an existing variety and a breeding process of the existing variety, response information including information pertaining to an existing variety that serves as a crossbreeding candidate for developing a new variety. More specifically, the generation section 405 generates response information based on the probability calculated by the link prediction section 403. A specific example of generation of the response information will be described later with reference to FIG. 15.

As described above, the link prediction section 403 carries out link prediction using the existing variety graph(s) that are each a learned model and the new variety graph that is generated based on a request. Therefore, the generation section 405 generates response information based on a result of link prediction by the link prediction section 403, and thereby generates response information based on the learned model and the request.

The basis generation section 406 generates basis information indicating validity of response information generated by the generation section 405. Specifically, the basis generation section 406 generates basis information including at least one selected from the group consisting of information pertaining to breeding of the existing variety that serves as a crossbreeding candidate and information pertaining to a character of the existing variety that serves as a crossbreeding candidate. The basis generation section 406 may generate basis information pertaining to a result of link prediction by the link prediction section 403 by analyzing the new variety graph and the existing variety graph(s).

The output section 407 outputs, for example, response information generated by the generation section 405. An output destination of the information is not particularly limited, as with the output section 208 in accordance with the second example embodiment.

As described above, the selective breeding assistance apparatus 4 includes the link prediction section 403 that calculates, by link prediction using the new variety graph and the existing variety graph(s), a probability that a node indicating a predetermined character links to a node included in the new variety graph. The generation section 405 generates response information based on the probability calculated by the link prediction section 403. A probability that a node indicating a predetermined character links to a new variety graph indicates a possibility that a new variety has the predetermined character, and response information generated based on the probability is useful in breeding of the new variety. Therefore, according to the configuration, it is possible to provide information useful for breeding of a new variety having an intended character.

### (Flow of Process)

The following description will discuss a flow of a process (selective breeding assistance method) carried out by the selective breeding assistance apparatus 4, with reference to FIG. 15. FIG. 15 is a flowchart illustrating the flow of the process carried out by the selective breeding assistance apparatus 4.

In S401, the reception section 401 receives a request pertaining to breeding. In S401, for example, a request is received which indicates a base variety which is a base of a new variety, a crossbreeding variety to be crossbred with the base variety, a character demanded of the new variety to be produced, and the like.

In S402, the graph generation section 402 generates a new variety graph based on the information input in S401. For example, in S401, upon receipt of input of a base variety and a crossbreeding variety, the graph generation section 402 may generate a new variety graph including nodes and links indicating that those varieties are parents of a new variety.

In S403, the link prediction section 403 calculates a probability that a node indicating a character that conforms to a request received in S401 links to a node included in the new variety graph generated in S402. As described above, calculation of the probability is carried out by link prediction using the learned existing variety graph(s) and the new variety graph. In association with the process of S403, the basis generation section 406 may analyze the new variety graph and the existing variety graph(s) to generate basis information indicating a basis of the calculation result by the link prediction section 403.

In S404, the graph updating section 404 determines whether or not the probability calculated in S403 is equal to or more than a threshold. If it has been determined that the probability is equal to or more than the threshold (YES in S404), the process proceeds to S406. If it has been determined that the probability is less than the threshold (NO in S404), the process proceeds to S405.

In a case where a plurality of characters are indicated in the request received in S401, prediction is carried out for each of the characters in S403. Then, in S404, the determination may be YES if probabilities for all of the characters are equal to or more than the threshold, and the determination may be NO if any one of the probabilities is less than the threshold. According to the configuration, it is possible to infer a crossbreeding variety that can produce a new variety having all of the requested characters.

In S405, the graph updating section 404 updates the new variety graph. Specifically, the graph updating section 404 replaces a node of a crossbreeding variety included in the current new variety graph with a node of another variety. The graph updating section 404 may change the base variety. The update content may be decided according to input by the user, or may be decided by the graph updating section 404, as described above.

When the new variety graph has been updated, the process returns to S403, and calculation of a probability is carried out again. That is, in the process of FIG. 15, until it is determined to be YES in S404, calculation of a probability in S403 and updating of the new variety graph in S405 are repeatedly carried out.

In S406, the generation section 405 infers a crossbreeding parent for producing a new variety that conforms to the request received in S401, and generates response information indicating the inferred crossbreeding parent. Specifically, the generation section 405 infers that a crossbreeding parent shown in the new variety graph at the time when it is determined to be YES in S404 is a crossbreeding parent for producing a new variety that conforms to the request, and generates response information indicating that crossbreeding parent. The crossbreeding parent to be inferred may be one parent (which is not a base variety) only or may be both parents.

In S407, the basis generation section 406 generates basis information indicating validity of the response information generated in S406. Specifically, the basis generation section 406 generates basis information including at least one selected from the group consisting of information pertaining to breeding of the existing variety that serves as a crossbreeding candidate and information pertaining to a character of the existing variety that serves as a crossbreeding candidate.

In S408, the output section 407 outputs the response information which has been generated in S406. At this time, the output section 407 may output also the basis information which has been generated in S407. Thus, the process of FIG. ends.

The selective breeding assistance method in accordance with the present example embodiment can also be utilized for breeding over a plurality of generations. In this case, after deciding a crossbreeding candidate as described above, the new variety graph may be updated to include a node indicating an offspring produced by crossbreeding of the varieties, and a crossbreeding candidate of that offspring may be decided. By repeating such a process until a new variety having an intended character is detected, it is possible to carry out assistance for production of even a new variety which is difficult to produce by a single crossbreeding.

### Fifth Example Embodiment

### (Overview)

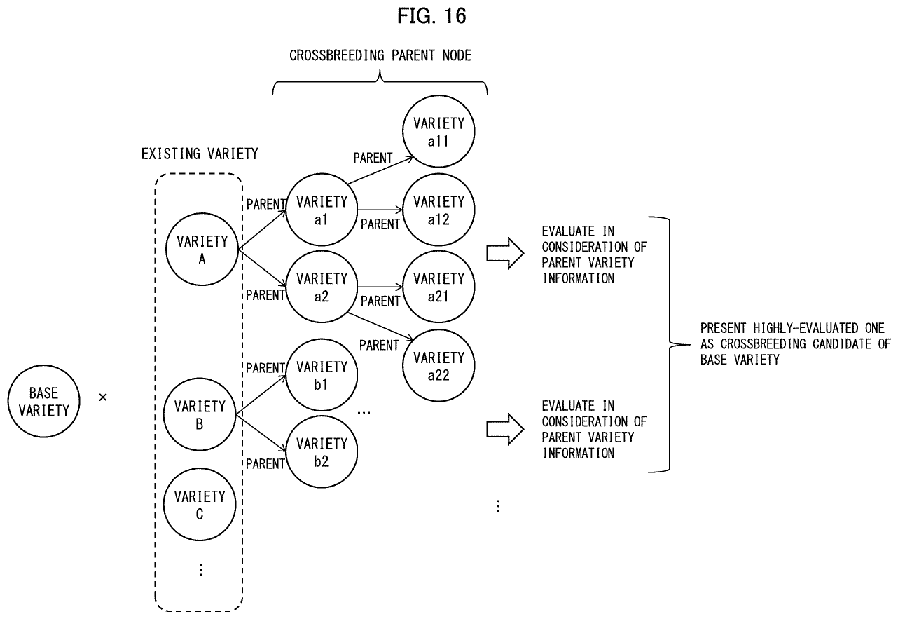

FIG. 16 is a diagram illustrating an overview of a selective breeding assistance method in accordance with the present example embodiment. In the present example embodiment, an example will be described in which suitability of an existing variety as a crossbreeding parent is evaluated using an existing variety graph including at least a crossbreeding parent node indicating a crossbreeding parent of the existing variety, and response information is generated based on the evaluation result.

An existing variety graph of a variety A illustrated in

FIG. 16 indicates that parents of the variety A are varieties a1 and a2, parents of the variety a1 are varieties a11 and a12, and parents of the variety a2 are varieties a21 and a22. Such a node indicating a crossbreeding parent of an existing variety is the crossbreeding parent node described above.

In the selective breeding assistance method in accordance with the present example embodiment, suitability of an existing variety as a crossbreeding parent is evaluated based on such a crossbreeding parent node. For example, in a case of evaluating suitability of the existing variety A illustrated in FIG. 16 as a crossbreeding parent to be crossbred with the base variety, evaluation is carried out based on the crossbreeding parent nodes (varieties a1, a2, a11, a12, a21, and a22) of the existing variety A.

Specifically, although not illustrated in FIG. 16, each of the crossbreeding parent nodes includes nodes and links indicating a character and the like of that variety. Therefore, the existing variety A may be evaluated based on those nodes and links.

A criterion for the above evaluation may be set in advance. For example, evaluation may be carried out on the basis of suitability for a user request pertaining to breeding. For example, it is assumed that a character demanded of a new variety to be produced is requested. In this case, it can be said that, if a node indicating the character is linked to a crossbreeding parent node, the character may be carried over to an offspring generation as well. Therefore, an existing variety in which a node indicating the requested character is linked to a crossbreeding parent node may be evaluated higher than an existing variety including no such link to a crossbreeding parent node.

Further, it can be said that, if a node indicating that a good character of a parental generation is carried over is linked to a crossbreeding parent node, there is a possibility that the good character of the parental generation is carried over also to an offspring generation. Therefore, an existing variety in which a node indicating that the good character of the parental generation is carried over is linked to a crossbreeding parent node may be evaluated higher than an existing variety including no such link to a crossbreeding parent node.

By evaluating each existing variety as described above, it is possible to infer an existing variety that is highly evaluated, i.e., an existing variety that should be a crossbreeding parent of a new variety, and to generate and output response information indicating the inference result. As such, according to the selective breeding assistance method in accordance with the present example embodiment, it is possible to generate response information useful for breeding of a new variety, while taking into consideration information pertaining to a crossbreeding parent of an existing variety.

In particular, in the selective breeding assistance method in accordance with the present example embodiment, it is possible, by using an existing variety graph, to trace crossbreeding strains in multiple stages (e.g., not only parents of an existing variety serving as a crossbreeding candidate but also grandparents and great-grandparents thereof), and to evaluate a crossbreeding candidate while taking into consideration those crossbreeding strains. According to the configuration, it is possible to carry out proper evaluation of each existing variety, and generate and output proper response information based on the proper evaluation result.

FIG. 16 illustrates the example of evaluating an existing variety to be crossbred with the base variety. Note, however, that an existing variety serving as a base variety can be evaluated in a similar manner. Thus, it is possible to identify also an existing variety suitable as a base variety.

### (Apparatus Configuration)

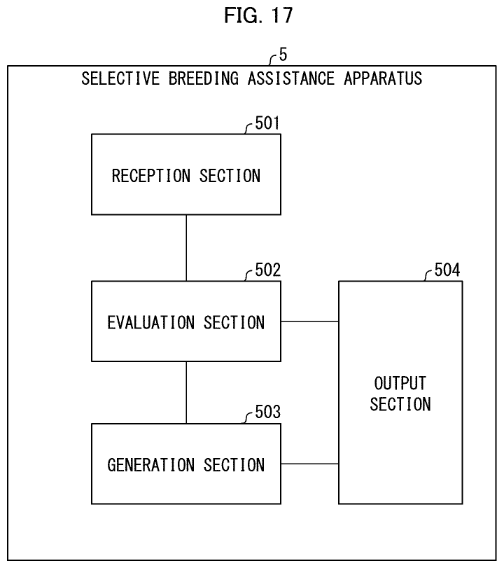

The following description will discuss a configuration of a selective breeding assistance apparatus 5 in accordance with the fifth example embodiment of the present invention, with reference to FIG. 17. FIG. 17 is a block diagram illustrating the configuration of the selective breeding assistance apparatus 5 in accordance with the present example embodiment.

As illustrated in FIG. 17, the selective breeding assistance apparatus 5 includes a reception section 501, an evaluation section 502, a generation section 503, and an output section 504. As with the selective breeding assistance apparatus 2 in accordance with the second example embodiment, the selective breeding assistance apparatus 5 may include, in addition to those constituent elements, a learning section 203, a basis generation section 207, an input apparatus, an output apparatus, a communication apparatus, and the like.

The reception section 501 receives a request pertaining to breeding. The request may include a user demand pertaining to breeding such as a character demanded of a new variety to be produced. The request may include information indicating a base variety.

The evaluation section 502 evaluates, for each of existing varieties that serve as crossbreeding candidates for use in production of a new variety, suitability as a crossbreeding parent of that existing variety based on a crossbreeding parent node of that existing variety. As described with reference to FIG. 16, various evaluation methods can be applied. Evaluation may be carried out while taking into consideration a request, or evaluation may be carried out without taking into consideration a request.

The evaluation section 502 may carry out the above evaluation while taking into consideration also an element other than the crossbreeding parent node (e.g., various kinds of information pertaining to the base variety). For example, the evaluation section 502 may evaluate an existing variety having a crossbreeding parent having a character which is not in the base variety higher than an existing variety which does not have a crossbreeding parent having such a character. The evaluation section 502 may carry out evaluations based on a plurality of criteria, integrate results of the evaluations based on the respective criteria, and use the integrated results as a final evaluation result.

The generation section 503 generates response information based on the evaluation result by the evaluation section 502. For example, the generation section 503 may generate response information indicating an existing variety evaluated by the evaluation section 502 and an evaluation result of the existing variety. For example, the generation section 503 may infer that a predetermined number of existing varieties which have been evaluated higher by the evaluation section 502 are existing varieties that each should be a crossbreeding parent of a new variety, and generate response information indicating the inference result.

In a case where the evaluation section 502 carries out evaluation without taking into consideration a request, the generation section 503 generates response information while taking into consideration the request. For example, the generation section 503 may infer that, among existing varieties which have been evaluated higher by the evaluation section 502, an existing variety that has high suitability for the request is an existing variety that should be a crossbreeding parent of the new variety. The generation section 503 may generate response information indicating the inference result. The suitability for the request may be determined based on a node which is directly or indirectly linked to the existing variety.

The output section 504 outputs, for example, response information generated by the generation section 503. An output destination of the information is not particularly limited, as with the output section 208 in accordance with the second example embodiment.

As described above, the selective breeding assistance apparatus 5 uses, as a learned model which has learned a relation between a property of an existing variety and a breeding process of the existing variety, an existing variety graph including at least a crossbreeding parent node indicating a crossbreeding parent of the existing variety. The selective breeding assistance apparatus 5 includes the evaluation section 502 that evaluates, based at least on the crossbreeding parent node, suitability as a crossbreeding parent of the existing variety, and the generation section 503 generates response information based on an evaluation result by the evaluation section 502. Thus, it is possible to generate response information useful for breeding of a new variety, while taking into consideration information pertaining to a crossbreeding parent of an existing variety.

### (Flow of Process)

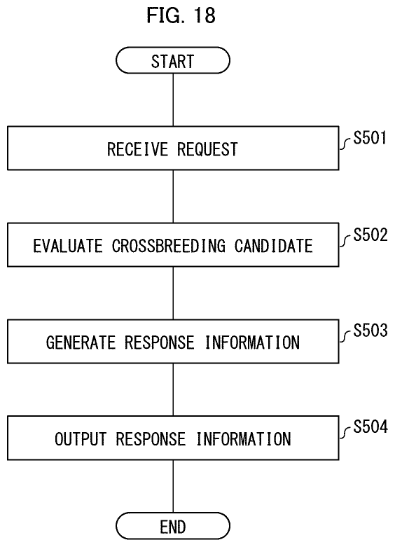

The following description will discuss a flow of a process (selective breeding assistance method) carried out by the selective breeding assistance apparatus 5, with reference to FIG. 18. FIG. 18 is a flowchart illustrating the flow of the process carried out by the selective breeding assistance apparatus 5.

In S501, the reception section 501 receives a request pertaining to breeding. In S501, for example, a request is received which includes a user demand pertaining to breeding such as a character demanded of a new variety to be produced.

In S502, the evaluation section 502 evaluates a crossbreeding candidate. More specifically, the evaluation section 502 evaluates, for each of existing varieties that serve as crossbreeding candidates for use in production of a new variety, suitability as a crossbreeding parent of that existing variety based on a crossbreeding parent node of that existing variety.

In S503, the generation section 503 generates response information based on the evaluation result in S502. For example, the generation section 503 may infer that a predetermined number of existing varieties which have been evaluated higher in S502 are existing varieties that each should be a crossbreeding parent of a new variety, and generate response information indicating the inference result.

In a case where the selective breeding assistance apparatus 5 includes the basis generation section 207, basis information indicating validity of the response information generated in S503 is generated by the basis generation section 207. In this case, the basis generation section 207 generates basis information including at least one selected from the group consisting of information pertaining to breeding of the existing variety that serves as a crossbreeding candidate and information pertaining to a character of the existing variety that serves as a crossbreeding candidate.

In S504, the output section 504 outputs the response information which has been generated in S503. In a case where basis information has been generated as described above, the output section 504 may output the basis information as well. Thus, the process of FIG. 18 ends.

### [Variation]

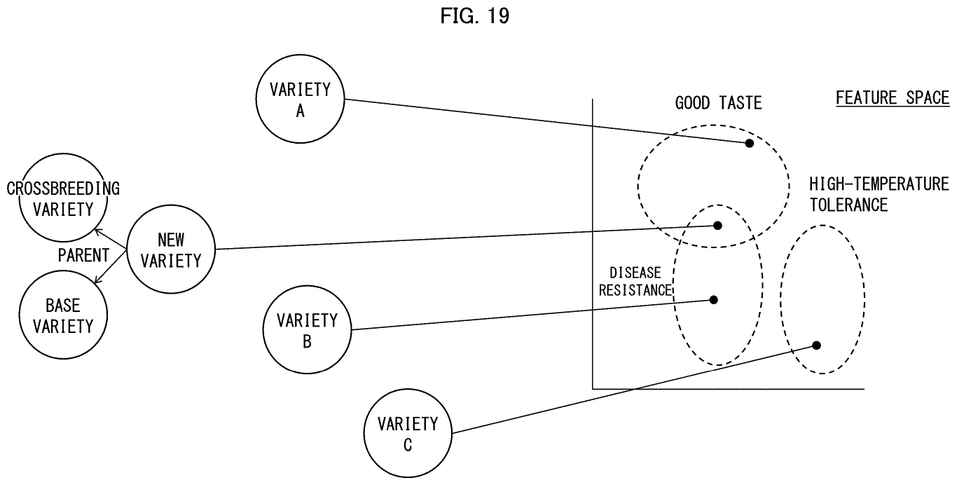

As described in the fourth example embodiment, by using a new variety graph and an existing variety graph, it is possible to predict, by link prediction, a character possessed by a new variety. Prediction of a character of the new variety can also be carried out by a method other than link prediction. This will be described with reference to FIG. 19. FIG. 19 is a diagram illustrating an example of predicting a character of a new variety based on a feature quantity calculated from a new variety graph and an existing variety graph. FIG. 19 shows existing variety graphs of existing varieties A through C and a new variety graph of a new variety. Note that, among nodes and links included in these graphs, those other than nodes and links indicating that parents of the new variety are a base variety and a crossbreeding variety are not illustrated.

Here, it is possible to calculate a feature quantity for each existing variety by multiplying a feature quantity of each node included in the existing variety graph by a weight corresponding to a link connected to that node, and adding up the weighted feature quantities. Therefore, by carrying out learning in which the weight is updated so that a calculated feature quantity corresponds to a character of the existing variety, it is possible to predict a character of a new variety from a feature quantity of a new variety graph which has been calculated while applying the weight.

For example, in the example of FIG. 19, a feature quantity calculated from the existing variety graph of the existing variety A that has been found to be good in taste is learned to fall within a range corresponding to a character “good taste” in a feature space. Moreover, a feature quantity calculated from the existing variety graph of the existing variety B that has been found to have disease resistance is learned to fall within a range corresponding to a character “disease resistance” in the feature space. Similarly, a feature quantity calculated from the existing variety graph of the existing variety C that has been found to have high-temperature tolerance is learned to fall within a range corresponding to a character “high-temperature tolerance” in the feature space.

In this case, if a feature quantity calculated from the new variety graph is included within a range corresponding to the characters “good taste” and “disease resistance” as illustrated in FIG. 19, it is possible to predict that the new variety has the characters “good taste” and “disease resistance”. Such a character prediction method can be applied as an alternative method to the method of character prediction in the above-described example embodiments.

### Software Implementation Example

Some or all of the functions of each of the selective breeding assistance apparatuses 1 through 5 (hereinafter, referred to as “present apparatus”) may be implemented by hardware such as an integrated circuit (IC chip), or may be implemented by software.

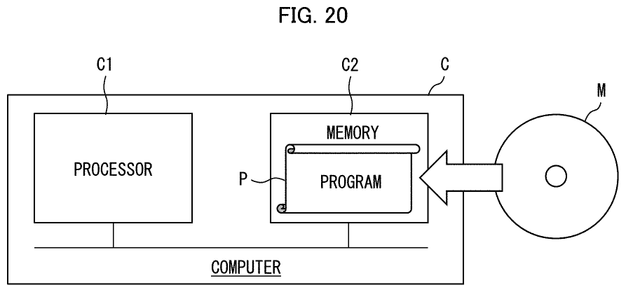

In the latter case, the present apparatus is realized by, for example, a computer that executes instructions of a program that is software realizing the foregoing functions. FIG. 20 illustrates an example of such a computer (hereinafter, referred to as “computer C”). The computer C includes at least one processor C1 and at least one memory C2. The memory C2 stores a program (selective breeding assistance program) P for causing the computer C to function as the present apparatus. In the computer C, the processor C1 reads the program P from the memory C2 and executes the program P, so that the functions of the present apparatus are realized.

Examples of the processor C1 include a central processing unit (CPU), a graphic processing unit (GPU), a digital signal processor (DSP), a micro processing unit (MPU), a floating point number processing unit (FPU), a physics processing unit (PPU), a microcontroller, and a combination thereof. Examples of the memory C2 include a flash memory, a hard disk drive (HDD), a solid state drive (SSD), and a combination thereof.

Note that the computer C can further include a random access memory (RAM) in which the program P is loaded when the program P is executed and in which various kinds of data are temporarily stored. The computer C can further include a communication interface for carrying out transmission and reception of data with other apparatuses. The computer C can further include an input-output interface for connecting input-output apparatuses such as a keyboard, a mouse, a display and a printer.

The program P can be stored in a computer C-readable, non-transitory, and tangible storage medium M. The storage medium M can be, for example, a tape, a disk, a card, a semiconductor memory, a programmable logic circuit, or the like. The computer C can obtain the program P via the storage medium M. The program P can be transmitted via a transmission medium. The transmission medium can be, for example, a communications network, a broadcast wave, or the like. The computer C can obtain the program P also via such a transmission medium.

### [Additional Remark 1]

The present invention is not limited to the foregoing example embodiments, but may be altered in various ways by a skilled person within the scope of the claims. For example, the present invention also encompasses, in its technical scope, any example embodiment derived by appropriately combining technical means disclosed in the foregoing example embodiments.

### [Additional Remark 2]

Some or all of the foregoing example embodiments can also be described as below. Note, however, that the present invention is not limited to the following supplementary notes.

### (Supplementary Note 1)

A selective breeding assistance apparatus, including: a reception means for receiving a request pertaining to breeding; a generation means for generating response information based on the request and a learned model which has learned a relation between a property of an existing variety and a breeding process of the existing variety, the response information including information pertaining to an existing variety that serves as a crossbreeding candidate for developing a new variety; and an output means for outputting the response information. According to the configuration, it is possible to bring about an example advantage of suitably assisting breeding.

### (Supplementary Note 2)

The selective breeding assistance apparatus according to supplementary note 1, in which: the request includes information indicating a character that is demanded of the new variety; and the selective breeding assistance apparatus further includes a basis generation means for generating basis information including at least one selected from the group consisting of information pertaining to breeding of the existing variety that serves as a crossbreeding candidate and information pertaining to a character of the existing variety that serves as a crossbreeding candidate. This configuration allows a user to refer to response information while taking into consideration basis information, and to precisely determine validity of the response information.

### (Supplementary Note 3)

The selective breeding assistance apparatus according to supplementary note 1 or 2, in which: the learned model is an existing variety graph which includes a plurality of nodes pertaining to the existing variety and a link indicating a relationship between the plurality of nodes and which has learned the relationship between the plurality of nodes. According to the configuration, it is possible to generate and output proper response information pertaining to an existing variety that serves as a crossbreeding candidate, while taking into consideration a mutual relationship between a crossbreeding result, parents, grandparents, a character, and the like of the existing variety.

### (Supplementary Note 4)

The selective breeding assistance apparatus according to supplementary note 3, further including: a link prediction means for predicting, by link prediction using a base variety graph and the existing variety graph, an existing variety to be crossbred with a base variety, the base variety graph including a plurality of nodes pertaining to the base variety which is one of crossbreeding parents of a new variety to be bred, and the link prediction being carried out for predicting a relationship between nodes which are not connected to each other by a link in the base variety graph and the existing variety graph, the generation means generating response information corresponding to the existing variety which has been predicted by the link prediction means. This makes it possible to recommend, to a user, an existing variety that seems to be proper as a crossbreeding candidate based on a past crossbreeding instance.

### (Supplementary Note 5)

The selective breeding assistance apparatus according to supplementary note 4, in which: the link prediction means predicts, as an existing variety to be crossbred with the base variety, an existing variety that satisfies at least one selected from the group consisting of a condition (1) that a variety which is obtained by crossbreeding that existing variety with an existing variety having a predetermined character maintains the predetermined character and a condition (2) of having a character to be added to the base variety.

In a case where an existing variety satisfying the condition (1) is predicted as an existing variety to be crossbred with the base variety, it is possible to predict, as an existing variety to be crossbred with the base variety, a variety that is less likely to lose a predetermined character when being crossbred with the base variety. In a case where an existing variety satisfying the condition (2) is predicted as an existing variety to be crossbred with the base variety, it is possible to predict, as an existing variety to be crossbred with the base variety, a variety that is more likely to express a character to be added to the base variety when being crossbred with the base variety.

### (Supplementary Note 6)

The selective breeding assistance apparatus according to supplementary note 4, further including: an evaluation means for evaluating, based on nodes included in an existing variety graph of the existing variety predicted by the link prediction means, suitability of the existing variety as a crossbreeding partner for the base variety. According to the configuration, it is possible to contribute to appropriate selection of a variety to be crossbred with the base variety.

### (Supplementary Note 7)

The selective breeding assistance apparatus according to supplementary note 3, further including: a link prediction means for identifying an existing variety that has a predetermined relationship with a new variety by link prediction using a new variety graph including a plurality of nodes pertaining to the new variety to be bred and existing variety graphs generated for a plurality of existing varieties, the generation means generating response information pertaining to the existing variety which has been identified by the link prediction means. According to the configuration, it is possible to provide information useful for breeding of a new variety.

### (Supplementary Note 8)

The selective breeding assistance apparatus according to supplementary note 3, further including: a link prediction means for identifying an existing variety that has a predetermined relationship with a base variety by link prediction using a base variety graph including a plurality of nodes pertaining to the base variety which is one of crossbreeding parents of a new variety to be bred and existing variety graphs generated for a plurality of existing varieties, the generation means generating response information pertaining to the existing variety which has been identified by the link prediction means. According to the configuration, it is possible to provide information useful for breeding using a base variety.

### (Supplementary Note 9)

The selective breeding assistance apparatus according to supplementary note 3, further including: a link prediction means for calculating, by link prediction using the existing variety graph and a new variety graph including a plurality of nodes related to a new variety to be bred, a probability that a node indicating a predetermined character links to a node included in the new variety graph, the generation means generating the response information based on the probability which has been calculated by the link prediction means. According to the configuration, it is possible to provide information useful for breeding of a new variety having an intended character.

### (Supplementary Note 10)

The selective breeding assistance apparatus according to supplementary note 1 or 2, in which: the learned model is an existing variety graph which at least includes a crossbreeding parent node indicating a crossbreeding parent of the existing variety; the selective breeding assistance apparatus includes an evaluation means for evaluating suitability of the existing variety as a crossbreeding parent based at least on the crossbreeding parent node; and the generation means generates the response information based on an evaluation result of the evaluation means. According to the configuration, it is possible to generate response information useful for breeding of a new variety, while taking into consideration information pertaining to a crossbreeding parent of an existing variety.

### (Supplementary Note 11)

A selective breeding assistance method, including: receiving, by a computer, a request pertaining to breeding; generating, by the computer, response information based on the request using a learned model which has learned a relation between a property of an existing variety and a breeding process of the existing variety, the response information including information pertaining to an existing variety that serves as a crossbreeding candidate for developing a new variety; and outputting, by the computer, the response information. According to the configuration, it is possible to bring about an example advantage of suitably assisting breeding.

### (Supplementary Note 12)

A selective breeding assistance program for causing a computer to carry out: a process of receiving a request pertaining to breeding; a process of generating response information based on the request using a learned model which has learned a relation between a property of an existing variety and a breeding process of the existing variety, the response information including information pertaining to an existing variety that serves as a crossbreeding candidate for developing a new variety; and a process of outputting the response information. According to the configuration, it is possible to bring about an example advantage of suitably assisting breeding.

### [Additional Remark 3]

Furthermore, some of or all of the foregoing example embodiments can also be expressed as below.

A selective breeding assistance apparatus including at least one processor, the at least one processor carrying out: a process of receiving a request pertaining to breeding; a process of generating response information based on the request using a learned model which has learned a relation between a property of an existing variety and a breeding process of the existing variety, the response information including information pertaining to an existing variety that serves as a crossbreeding candidate for developing a new variety; and a process of outputting the response information.

The selective breeding assistance apparatus may further include a memory. The memory may store a program (selective breeding assistance program) for causing the at least one processor to carry out: a process of receiving a request pertaining to breeding; a process of generating response information based on the request using a learned model which has learned a relation between a property of an existing variety and a breeding process of the existing variety, the response information including information pertaining to an existing variety that serves as a crossbreeding candidate for developing a new variety; and a process of outputting the response information. The program can be stored in a computer-readable non-transitory tangible storage medium.

### REFERENCE SIGNS LIST

1 Selective breeding assistance apparatus:
11 Reception section, 12 Generation section, 13 Output section
2 Selective breeding assistance apparatus:
201 Reception section, 204 Link prediction section, 205 Evaluation section, 206 Generation section, 207 Basis generation section, 208 Output section
3 Selective breeding assistance apparatus:
301 Reception section, 303 Link prediction section, 304 Evaluation section, 305 Generation section, 306 Basis generation section, 307 Output section
4 Selective breeding assistance apparatus:
401 Reception section, 403 Link prediction section, 405 Generation section, 406 Basis generation section, 407 Output section
5 Selective breeding assistance apparatus:
501 Reception section, 502 Evaluation section, 503 Generation section, 504 Output section

What is claimed is:

1. A selective breeding assistance apparatus, comprising at least one processor, the at least one processor carrying out:
a reception process of receiving a request pertaining to breeding;
a generation process of generating response information based on the request and a learned model which has learned a relation between a property of an existing variety and a breeding process of the existing variety, the response information including information pertaining to an existing variety that serves as a crossbreeding candidate for developing a new variety; and
an output process of outputting the response information.

2. The selective breeding assistance apparatus according to claim 1, wherein:
the request includes information indicating a character that is demanded of the new variety; and
the at least one processor further carries out a basis generation process of generating basis information including at least one selected from the group consisting of information pertaining to breeding of the existing variety that serves as a crossbreeding candidate and information pertaining to a character of the existing variety that serves as a crossbreeding candidate.

3. The selective breeding assistance apparatus according to claim 1, wherein:
the learned model is an existing variety graph which includes a plurality of nodes pertaining to the existing variety and a link indicating a relationship between the plurality of nodes and which has learned the relationship between the plurality of nodes.

4. The selective breeding assistance apparatus according to claim 3, wherein:
the at least one processor further carries out a link prediction process of predicting, by link prediction using a base variety graph and the existing variety graph, an existing variety to be crossbred with a base variety, the base variety graph including a plurality of nodes pertaining to the base variety which is one of crossbreeding parents of a new variety to be bred, and the link prediction being carried out for predicting a relationship between nodes which are not connected to each other by a link in the base variety graph and the existing variety graph; and
in the generation process, the at least one processor generates response information corresponding to the existing variety which has been predicted by in the link prediction process.

5. The selective breeding assistance apparatus according to claim 4, wherein:
in the link prediction process, the at least one processor predicts, as an existing variety to be crossbred with the base variety, an existing variety that satisfies at least one selected from the group consisting of a condition (1) that a variety which is obtained by crossbreeding that existing variety with an existing variety having a predetermined character maintains the predetermined character and a condition (2) of having a character to be added to the base variety.

6. The selective breeding assistance apparatus according to claim 4, wherein:
the at least one processor carries out an evaluation process of evaluating, based on nodes included in an existing variety graph of the existing variety predicted in the link prediction process, suitability of the existing variety as a crossbreeding partner for the base variety.

7. The selective breeding assistance apparatus according to claim 3, wherein:
the at least one processor further carries out a link prediction process of identifying an existing variety that has a predetermined relationship with a new variety by link prediction using a new variety graph including a plurality of nodes pertaining to the new variety to be bred and existing variety graphs generated for a plurality of existing varieties; and
in the generation process, the at least one processor generates response information pertaining to the existing variety which has been identified in the link prediction process.

8. The selective breeding assistance apparatus according to claim 3, wherein:
the at least one processor further carries out a link prediction process of identifying an existing variety that has a predetermined relationship with a base variety by link prediction using a base variety graph including a plurality of nodes pertaining to the base variety which is one of crossbreeding parents of a new variety to be bred and existing variety graphs generated for a plurality of existing varieties; and
in the generation process, the at least one processor generates response information pertaining to the existing variety which has been identified in the link prediction process.

9. The selective breeding assistance apparatus according to claim 3, wherein:
the at least one processor further carries out a link prediction process of calculating, by link prediction using the existing variety graph and a new variety graph including a plurality of nodes related to a new variety to be bred, a probability that a node indicating a predetermined character links to a node included in the new variety graph; and
in the generation process, the at least one processor generates the response information based on the probability which has been calculated in the link prediction process.

10. The selective breeding assistance apparatus according to claim 1, wherein:
the learned model is an existing variety graph which at least includes a crossbreeding parent node indicating a crossbreeding parent of the existing variety;
the at least one processor carries out an evaluation process of evaluating suitability of the existing variety as a crossbreeding parent based at least on the crossbreeding parent node; and
in the generation process, the at least one processor generates the response information based on an evaluation result of in the evaluation process.

11. A selective breeding assistance method, comprising:
receiving, by a computer, a request pertaining to breeding;
generating, by the computer, response information based on the request using a learned model which has learned a relation between a property of an existing variety and a breeding process of the existing variety, the response information including information pertaining to an existing variety that serves as a crossbreeding candidate for developing a new variety; and
outputting, by the computer, the response information.

12. A computer-readable non-transitory storage medium storing a selective breeding assistance program for causing a computer to carry out:
a process of receiving a request pertaining to breeding;
a process of generating response information based on the request using a learned model which has learned a relation between a property of an existing variety and a breeding process of the existing variety, the response information including information pertaining to an existing variety that serves as a crossbreeding candidate for developing a new variety; and
a process of outputting the response information.
